(19) United States
(12) Patent Application Publication
(10) Pub. No.: US 2026/0325487 A1
(43) Pub. Date: Oct. 22, 2026

(54) A PHARMACEUTICAL COMPOSITION COMPRISING 17-AAG AND STAUROSPORINE FOR USE IN THE PREVENTION OR TREATMENT OF CANCER AND DISEASES RESULTING FROM EXCESSIVE CELL PROLIFERATION AND A POLYMER- LIPID NANOPARTICLE CONTAINING THE COMPOSITION

(71) Applicant: BS BIOTECHNA SPOLKA AKCYJNA, Krakow (PL)

(72) Inventors: Olga SWIECH, Bieniewice (PL); Rafal MACKIEWICZ, Wyszegrod (PL); Weronika PIOTROWSKA, Warszawa (PL); Natalia LABEDZ; Mateusz SURSKI, Wroclaw (PL); Joanna WIETRZYK, Wroclaw (PL); Wojciech SMORON, Krakow (PL); Julia WITEK, Sieradz (PL)

(21) Appl. No.: 19/480,933

(22) PCT Filed: May 3, 2024
(86) PCT No.: PCT/PL2024/000021

§ 371 Date: Nov. 3, 2025

(30) Foreign Application Priority Data
May 3, 2023 (PL) P.444725

(51) Int. Cl. A61K 31/395; A61K 9/51; A61K 31/553; A61P 35/00

(52) U.S. Cl. CPC A61K 31/395; A61K 9/5123; A61K 9/5138; A61K 9/5153; A61K 31/553; A61P 35/00

(57) ABSTRACT

A composition comprising 17-AAG and staurosporine for use in the treatment of melanoma, non-small cell lung cancer, pancreatic cancer, mammary gland tumours or gliomas, and a stable polymer-lipid nanoparticle for drug binding and delivery, consisting of a core containing polylactic-co-glycolic acid (PLGA) and polyvinyl alcohol (PVA) and a core envelope containing a mixture of lipids: 1,2-dipalmitoyl-sn-glycero-3-phosphocholine (DDPC), cholesterol and the ammonium salt 1,2-distearoyl-sn-glycero-3-phosphoethanolamine-N-[amino (polyethylene glycol)-2000 (DSPE-PEG(2000)NH2J.

The object of the invention is a pharmaceutical composition for the prevention or treatment of cancer and diseases resulting from excessive cell proliferation, in particular melanoma, non-small cell carcinoma, lung cancer, pancreatic cancer, mammary gland tumours or gliomas, and a nanoparticle containing the composition.

Cancer treatment can be local or systemic. Local treatment usually consists of removing the tumour or using radiotherapy. Systemic treatment affects other cells in the body in addition to the tumour cells. Such methods include chemotherapy, hormone therapy or targeted treatment, which involves the administration of intravenous or oral drugs that are tailored to the progression of the disease. Other methods of cancer treatment are also known from the state of the art.

From the description of the invention US2004110662A1—the administration of heat shock protein inhibitors and cytotoxic agents in succession is known.

From the description of WO2021007512A1, the administration of an effective amount of a platinum-based chemotherapeutic agent in combination with a microtubule-associated serine/threonine protein kinase (MAST) inhibitor or a glucocorticoid receptor-binding agent or an hsp90-binding agent is known.

Description WO2009009067A-discloses the encapsulation of 17AAG or 17DMAG in PEG-PLA micelles and their use in combination with other active substances, such as anticancer or cytotoxic substances.

The essence of the pharmaceutical composition solution according to the invention is a composition comprising 17-AAG (tanespimycin) at a concentration of not less than 0.31 nM and staurosporine at a concentration of not less than 0.031 nM for use in the treatment of melanoma, non-small cell lung cancer, pancreatic cancer, mammary gland tumours or gliomas.

Favourably, the ratio of 17-AAG to staurosporine concentrations is between 1/100 and 1/1.

The essence of the solution according to the invention for the polymer-lipid nanoparticle is that the carrier consisting of a core containing polylactic-co-glycolic acid (PLGA) in an amount of 16.1 to 30.3 wt % and polyvinyl alcohol (PVA) in an amount of 60.6 to 75.5 wt %, and a core envelope, which is a mixture of lipids: 1,2-dipalmitoyl-sn-glycero-3-phosphocholine (DDPC) in an amount ranging from 3.66% to 12.6% by weight, cholesterol in an amount ranging from 1.85 to 6.29% by weight and the ammonium salt of 1,2-distearoyl-sn-glycero-3-phosphoethanolamine-N [amino (polyethylene glycol)-2000 (DSPE-PEG (2000)NH2] in an amount of 0.15 to 0.5% by weight contains a pharmaceutical composition comprising 17-AAG (tanespimycin) in a concentration of not less than 0.31 nM and staurosporine at a concentration of not less than 0.031 nM.

Favourably, the ratio of 17-AAG to staurosporine concentrations is between 100/1 and 1/1.

Advantageously, PLGA is a 50:50 copolymer with an Mw of 24 000-38 000 g/mol.

Advantageously, PVA has a mass Mw of 30 000-70 000 g/mol.

The main advantage of the solutions according to the inventions is that the composition used has a dual action, leading to the arrest of growth or death of the cell by synergistically combining two mechanisms that would generally not be targeted by a single inhibitor, leading to the arrest of growth or death of the cancer cell. The synergism occurring with staurosporine and 17-AAG allows the use of much lower doses of the substances and reduced side effects of their use. The administration of the therapeutic composition in the form of a nanocapsule enables its use in molecularly targeted therapy.

This favourably influences the effect of the therapy, significantly increasing its efficacy and minimising the negative impact on other cells and negative effects occurring in classical anti-cancer therapies. The applied nanoparticle allows loading of the pharmaceutical composition at a level of 32% to 80%, long-term release of drug combinations and prevention of excessive drug leakage and carrier aggregation in the presence of plasma proteins. In addition, it allows a range of 17AAG to Staurosporine concentration ratios from 0.62 to 9.5. The applied lipid bilayer coating with polyethyleneglycol prevents nanoparticle aggregation in the presence of proteins and ensures nanoparticle stability at 37° C. over 96 h. The size of the PLGA/PVA/LIPO nanoparticles, ranging from 209 nm to 388 nm, ensures selective, passive transport of the drug into tumour tissues due to the effect of increased permeability and EPR retention. In addition, the additional modification of polyethylene glycol with amine groups giving the nanoparticles a low positive potential of 6.5 mV to 9.5 mV allows for increased anti-tumour efficacy, better accumulation and penetration of the carrier in tumour tissues and better cellular uptake compared to inert and negatively charged nanoparticles (https://doi.org/10.1016/j.nantod.2016.04.008). With such a developed structure, the developed nanoparticles have a small diameter and low PDI polydispersity index.

The solutions according to the inventions are illustrated by the following manufacturing examples and drawings, where:

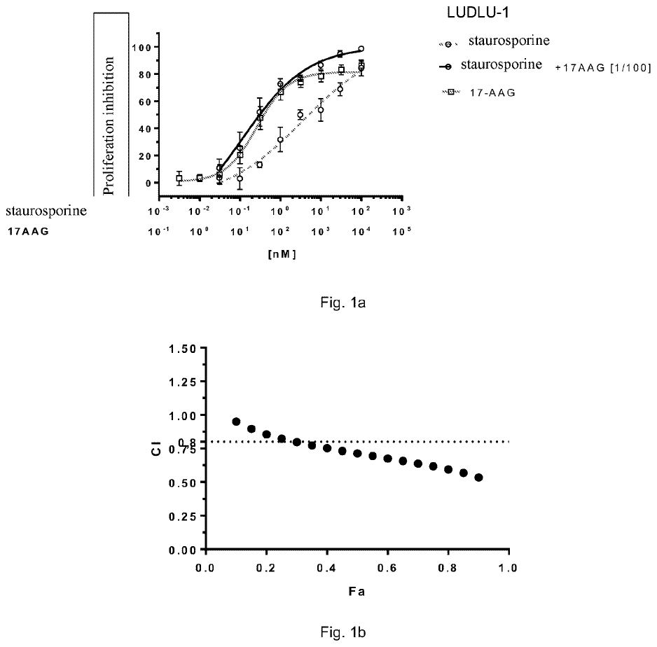

FIG. 1a—Growth kinetics of human lung cancer tumours LUDLU-1 in mice treated with Staurosporine, 17AAG and their combination,

FIG. 1b—Fa-Cl plot,

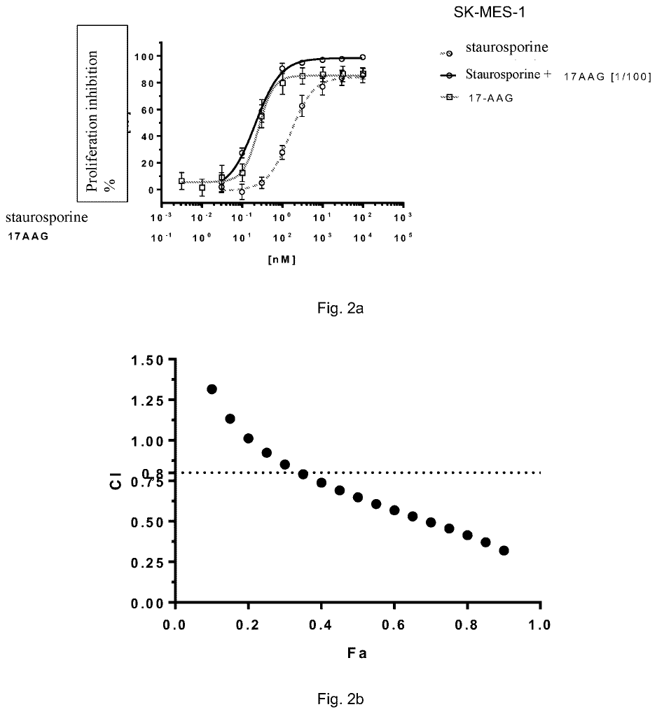

FIG. 2a-Growth kinetics of human lung cancer tumours SK-MES-1 in mice treated with Staurosporine,

FIG. 2b—Fa-Cl plot,

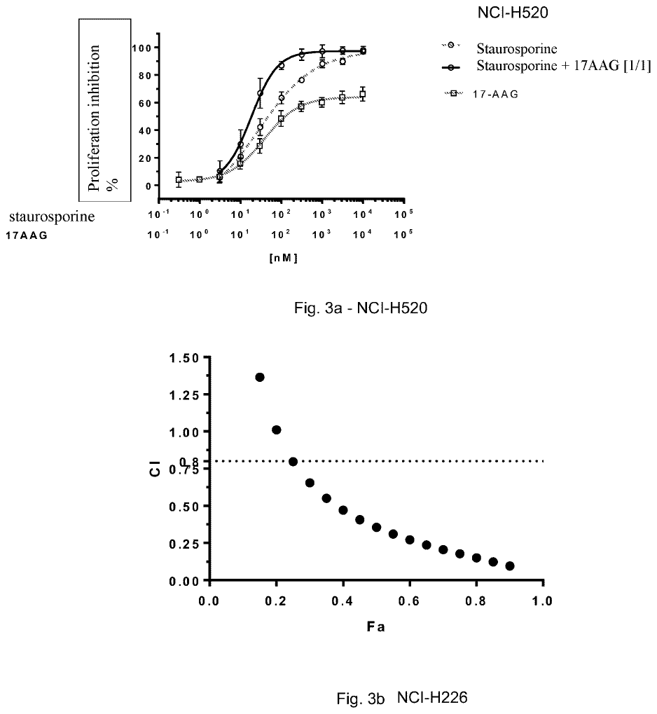

FIG. 3a—Growth kinetics of human lung cancer tumours NCI-H520 in mice treated with Staurosporine,

FIG. 3b—Fa-Cl plot,

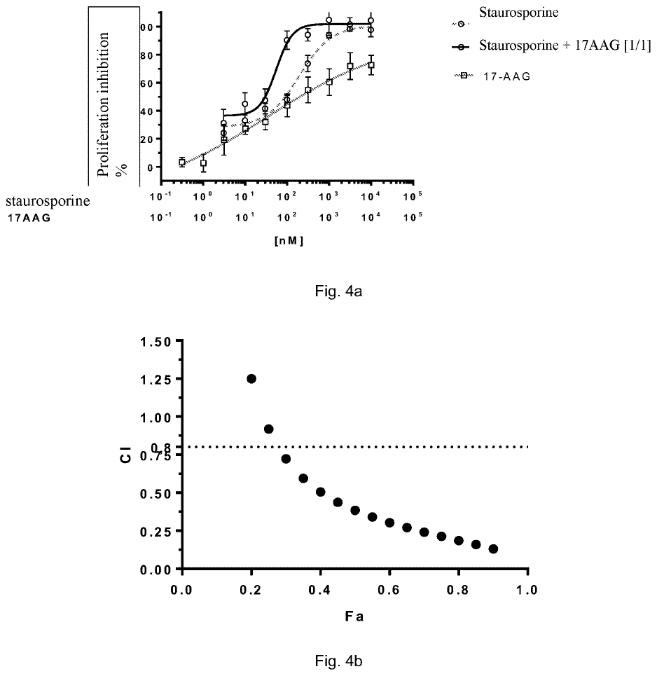

FIG. 4a—Growth kinetics of human lung cancer tumours NCI-H226 in mice treated with Staurosporine,

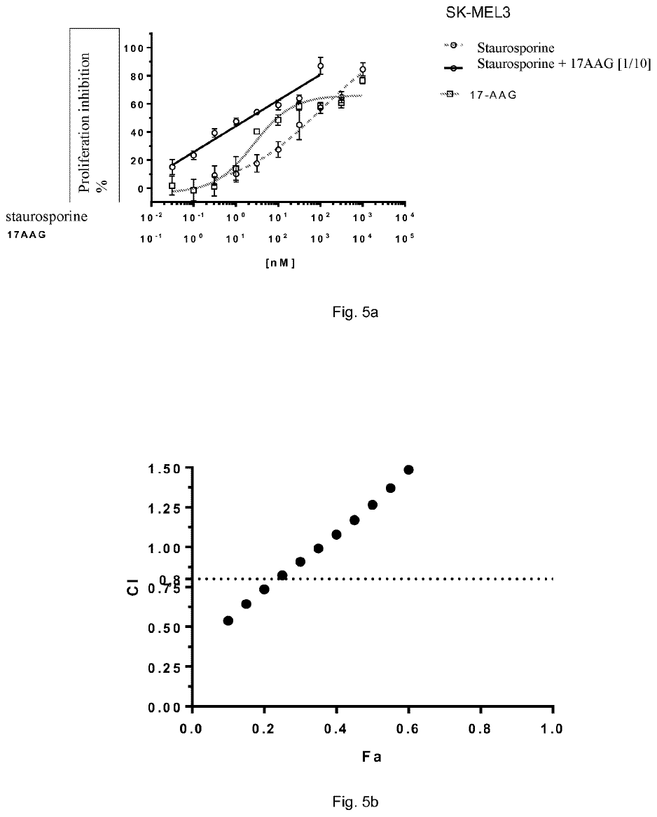

FIG. 4b—Fa-Cl plot, FIG. 5a—Growth kinetics of human lung cancer tumours NCI-H226 in mice treated with Staurosporine,

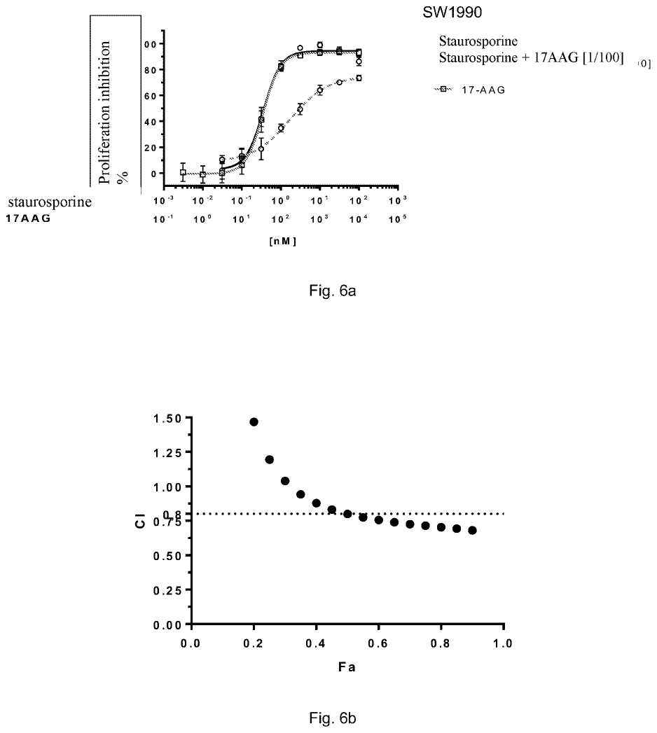

FIG. 5b—Fa-Cl plot, FIG. 6a-Growth kinetics of human lung cancer tumours SW1990 in mice treated with Staurosporine,

FIG. 6b—Fa-Cl plot,

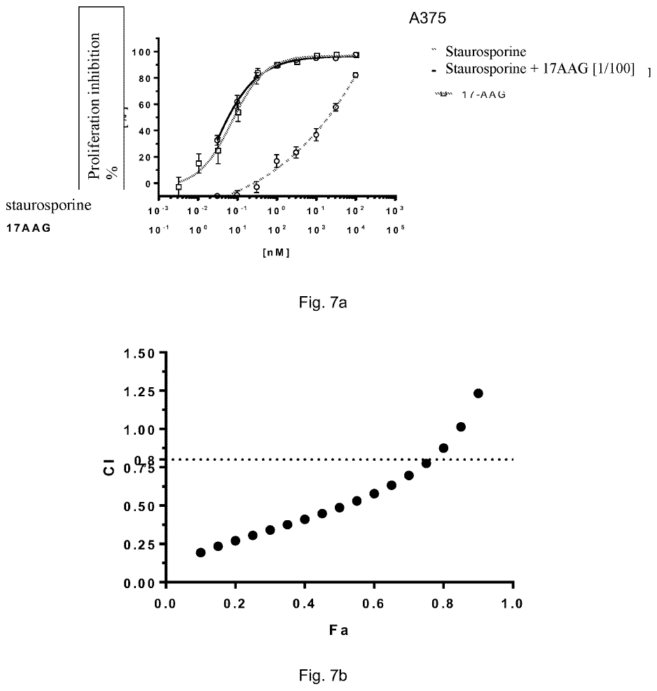

FIG. 7a—Growth kinetics of A375 human lung cancer tumours in Staurosporine-treated mice,

FIG. 7b—Fa-Cl plot,

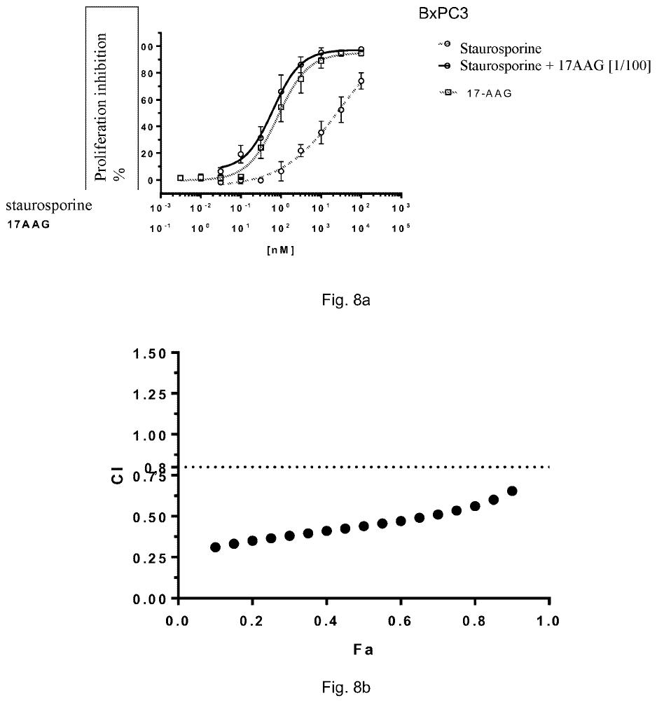

FIG. 8a—Growth kinetics of BxPC3 human lung cancer tumours in Staurosporine-treated mice,

FIG. 8b—Fa-Cl plot,

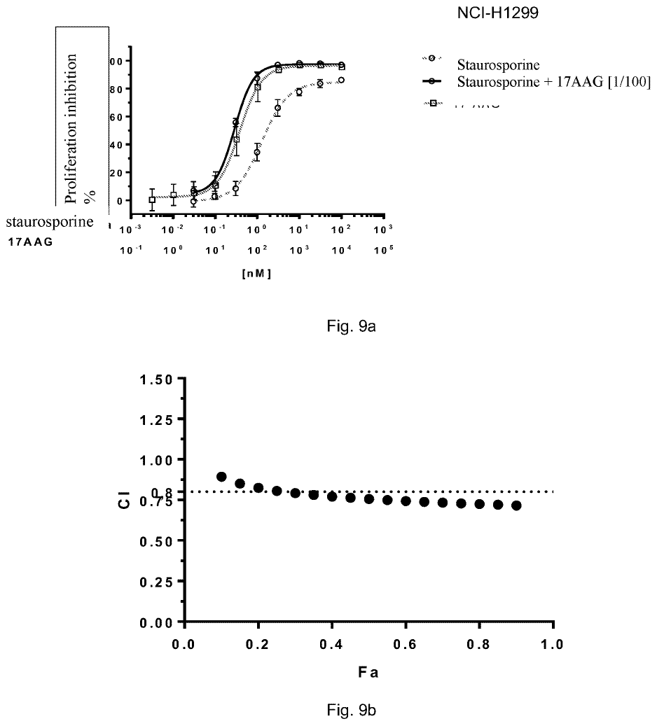

FIG. 9a—Growth kinetics of NCI-H1299 human lung cancer tumours in Staurosporine-treated mice,

FIG. 9b—Fa-Cl plot, 10a—Growth kinetics of human lung cancer tumours NCI-H1975 in mice treated with Staurosporine,

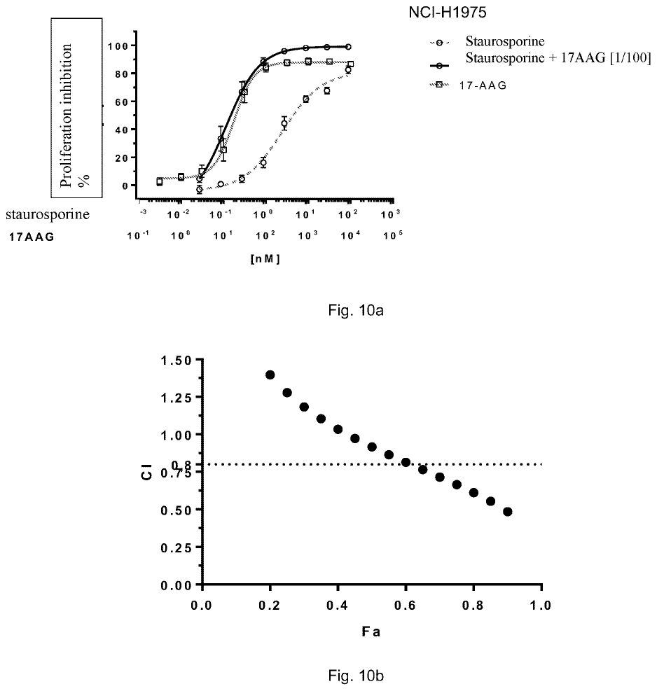

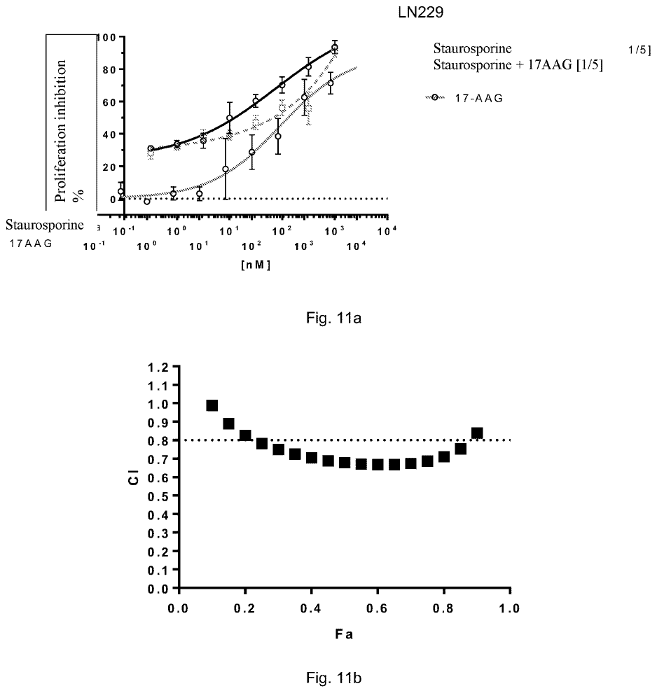

FIG. 10b—Fa-Cl plot, FIG. 11a—Growth kinetics of human lung cancer tumours LN229 in mice treated with Staurosporine,

FIG. 11b—Fa-Cl plot,

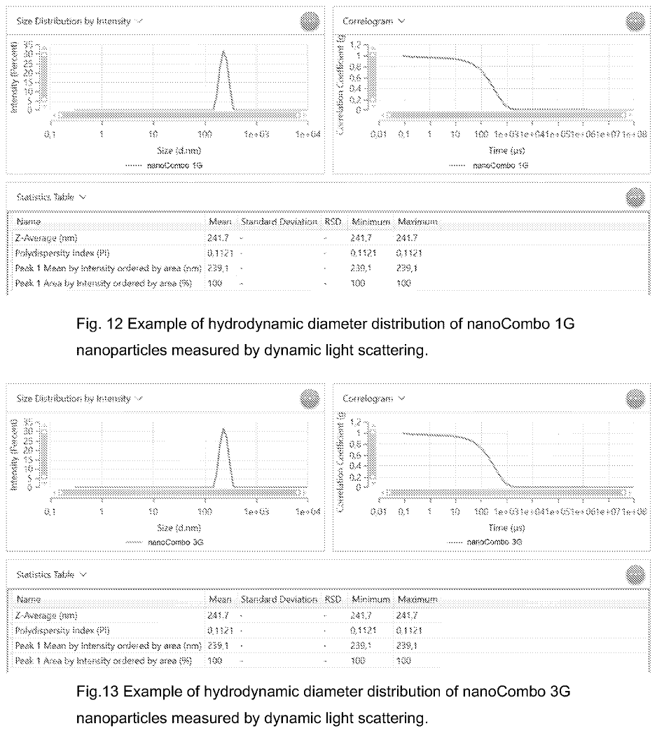

FIG. 12—Example of hydrodynamic diameter distribution of nanoCombo 1G nanoparticles measured by dynamic light scattering,

FIG. 13 Example distribution of hydrodynamic diameters of nanoCombo 3G nanoparticles measured by dynamic light scattering,

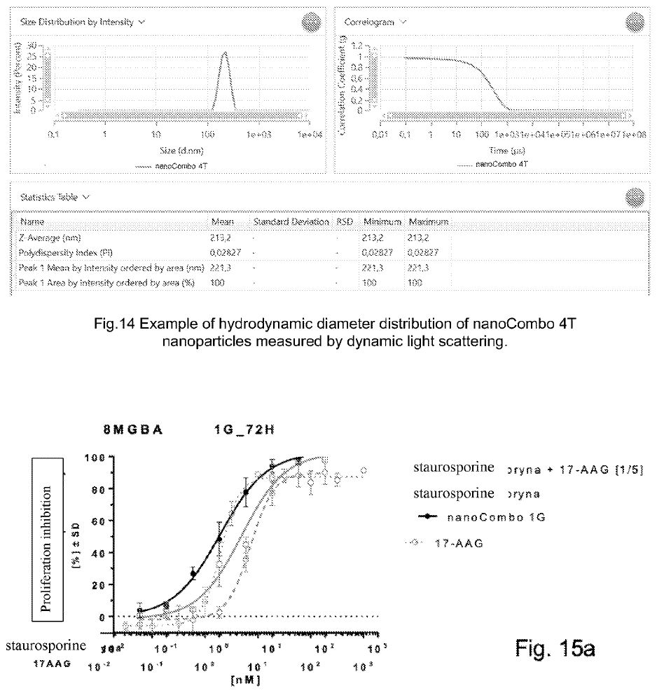

FIG. 14—Example distribution of hydrodynamic diameters of nanoCombo 4T nanoparticles measured by dynamic light scattering,

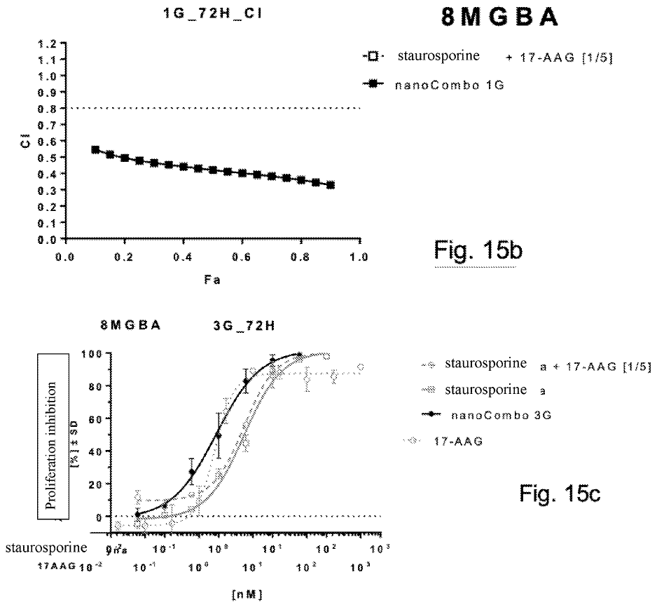

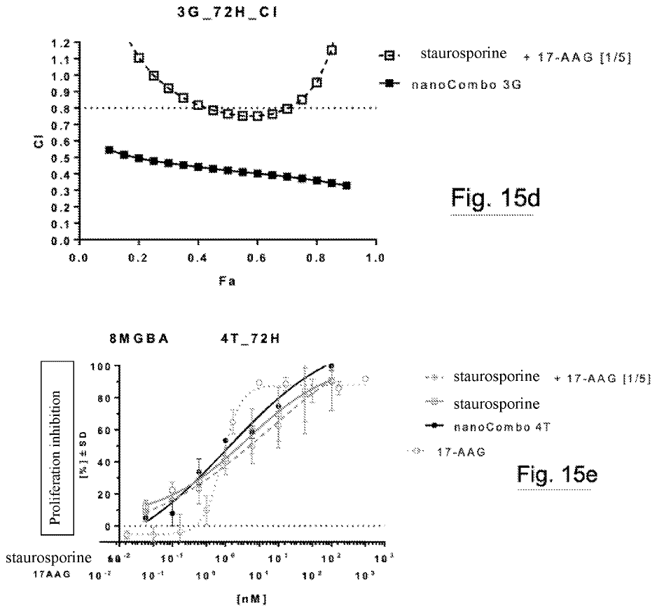

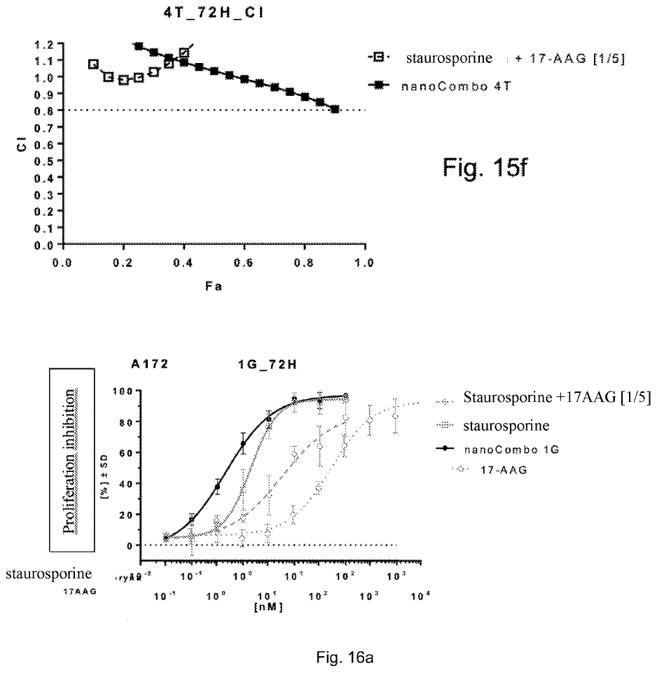

FIG. 15A, C, E—Growth kinetics of 8MGBA human lung cancer tumours in mice treated with Staurosporine for different nanoparticles,

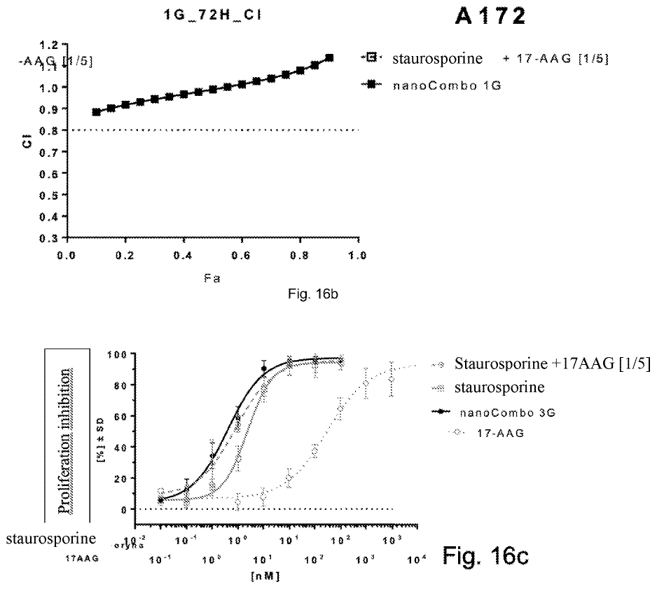

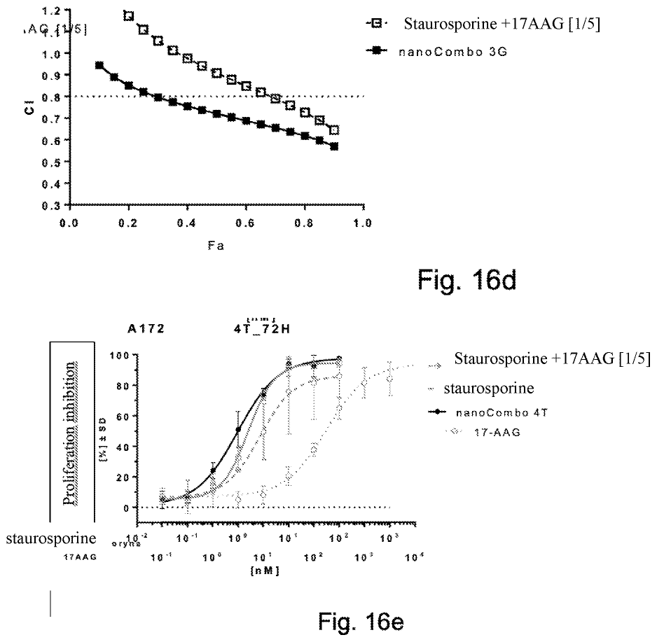

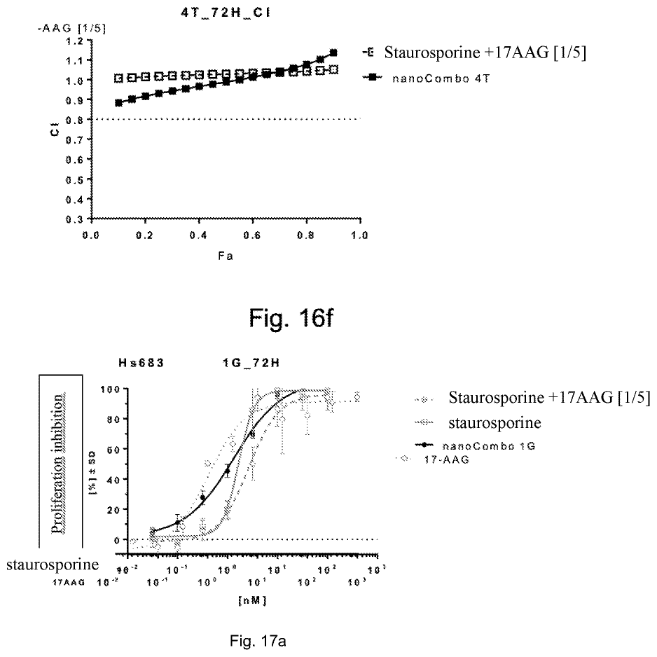

FIG. 16A, C, E—Growth kinetics of A172 human lung cancer tumours in mice treated with Staurosporine for different nanoparticles,

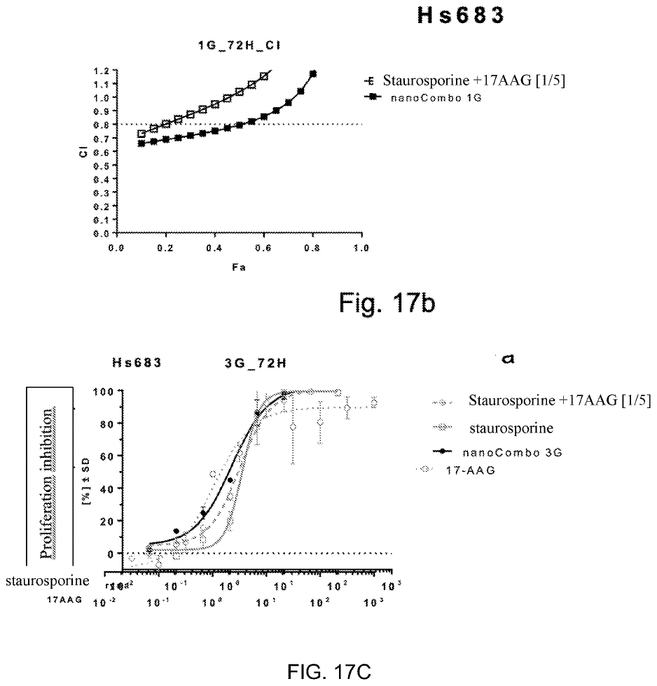

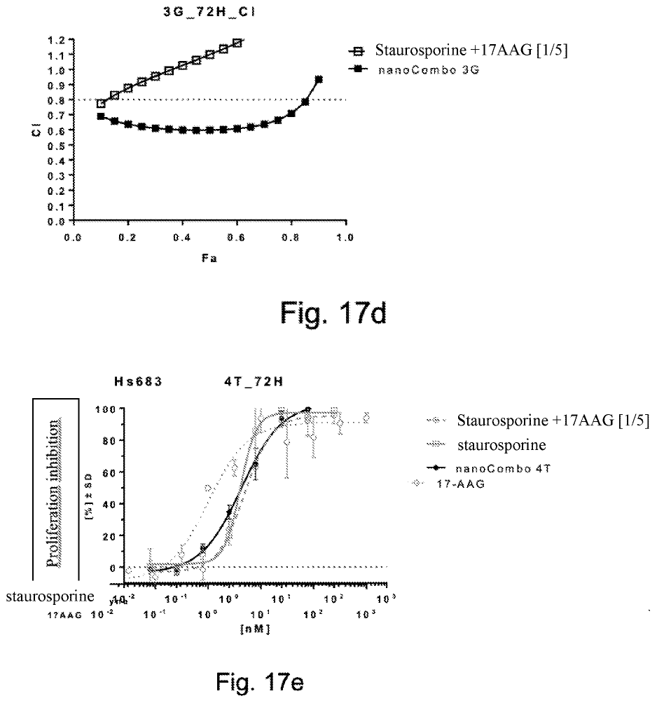

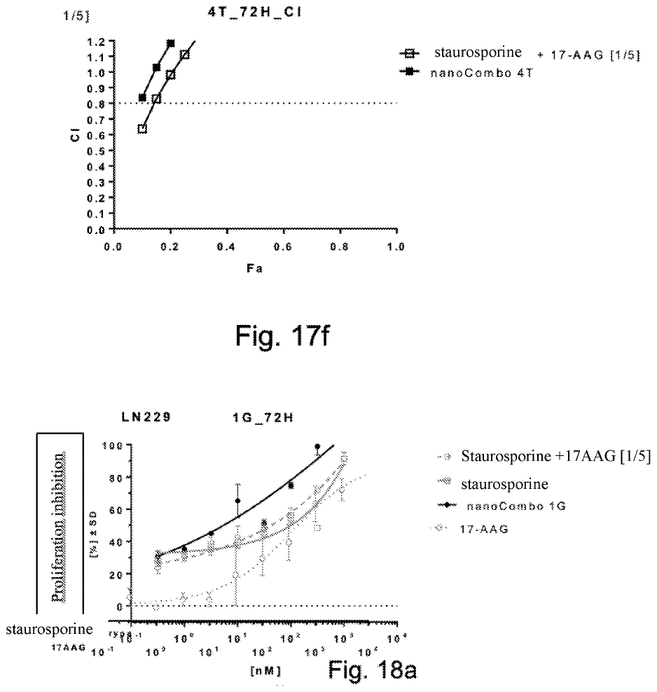

FIG. 17A, C, E—Growth kinetics of HS683 human lung cancer tumours in mice treated with Staurosporine for different nanoparticles,

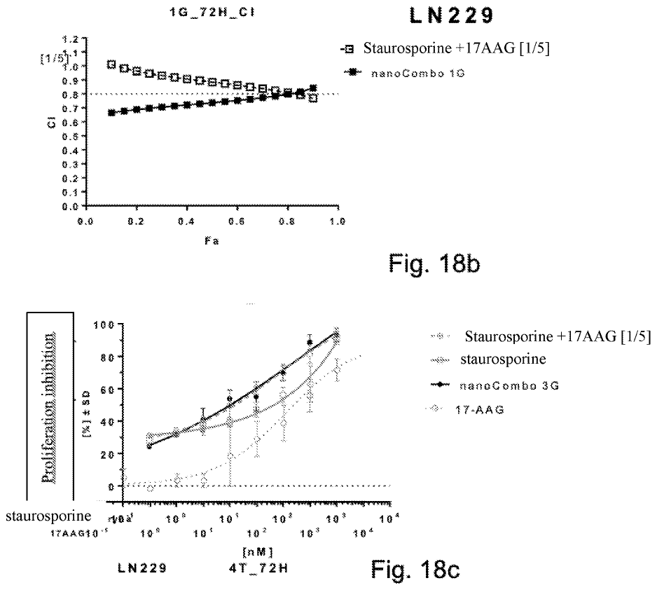

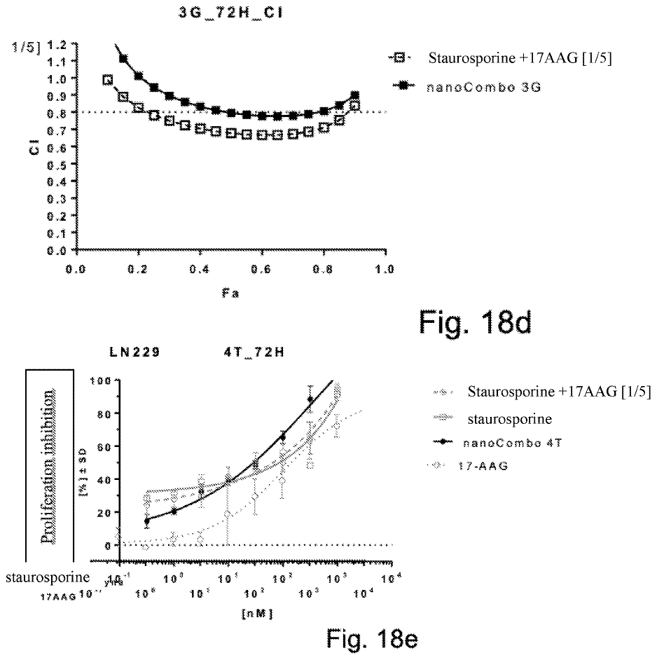

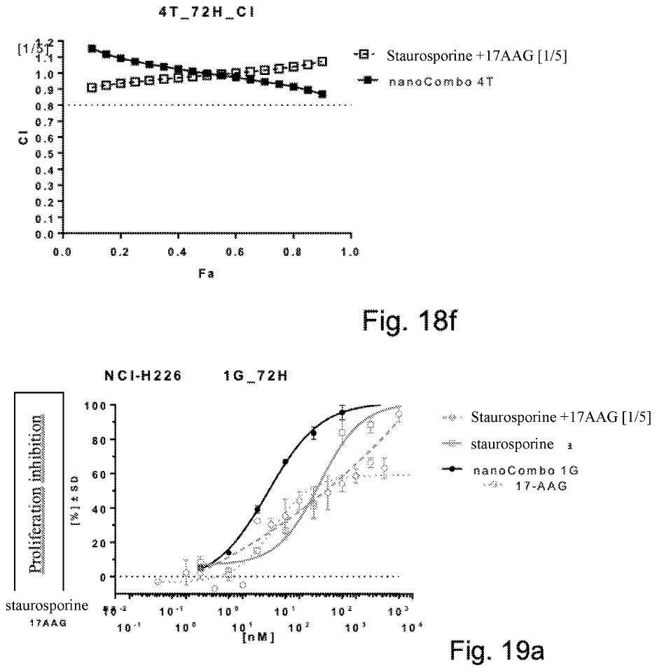

FIG. 18A, C, E—Growth kinetics of human lung cancer tumours LN229 in mice treated with Staurosporine for different nanoparticles,

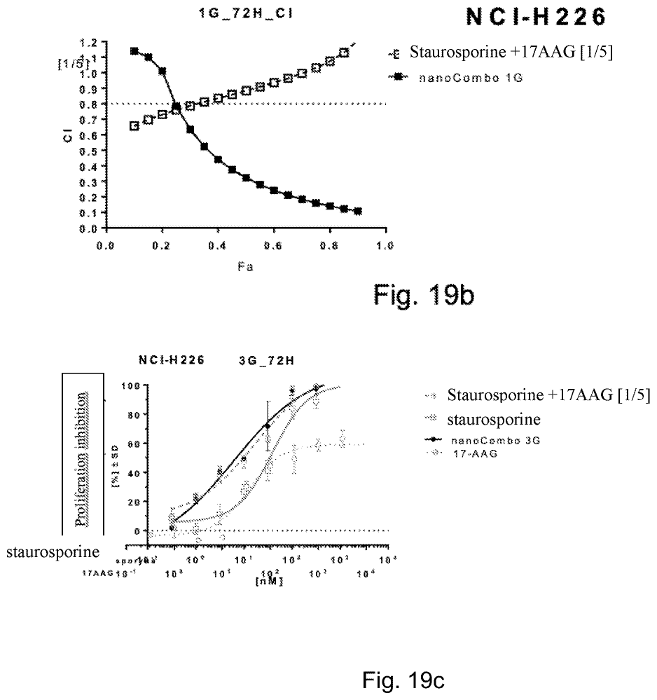

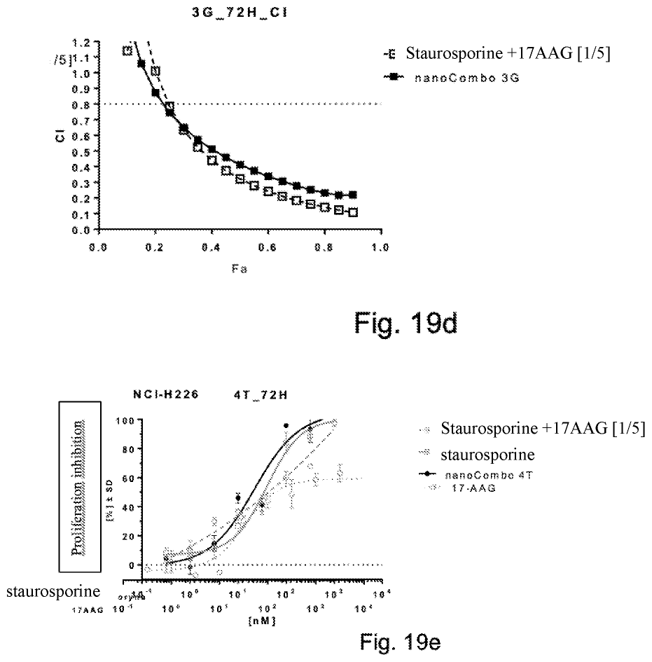

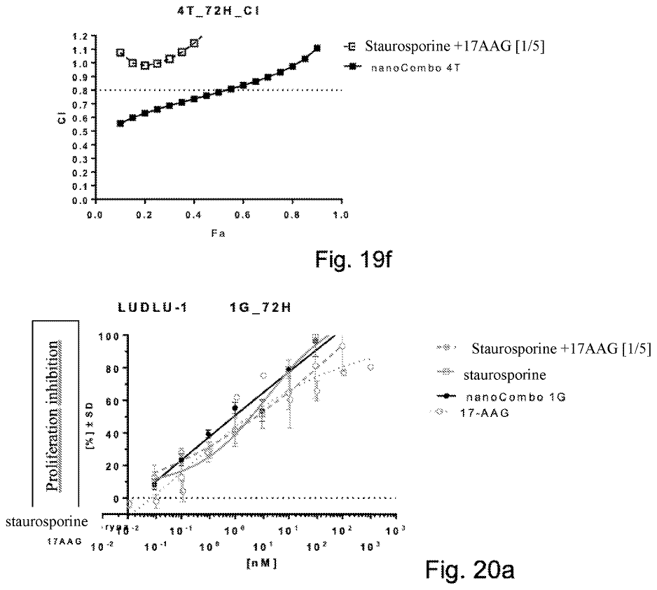

FIG. 19A, C, E—Growth kinetics of human lung cancer tumours HS683 in mice treated with Staurosporine for different nanoparticles,

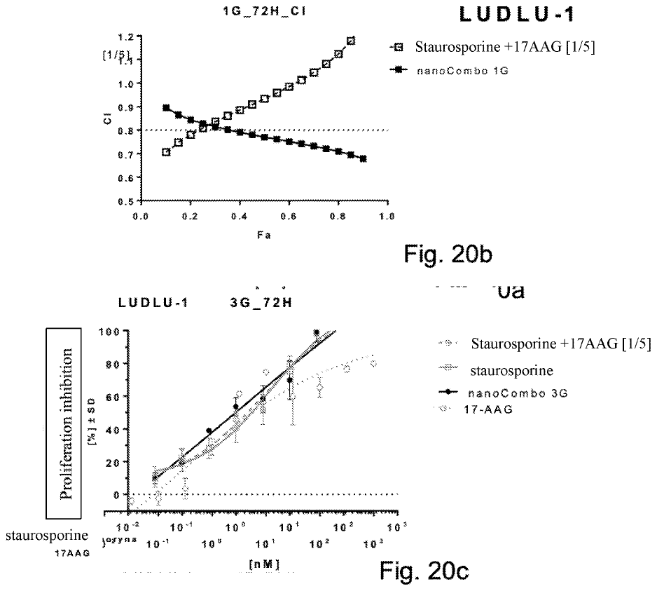

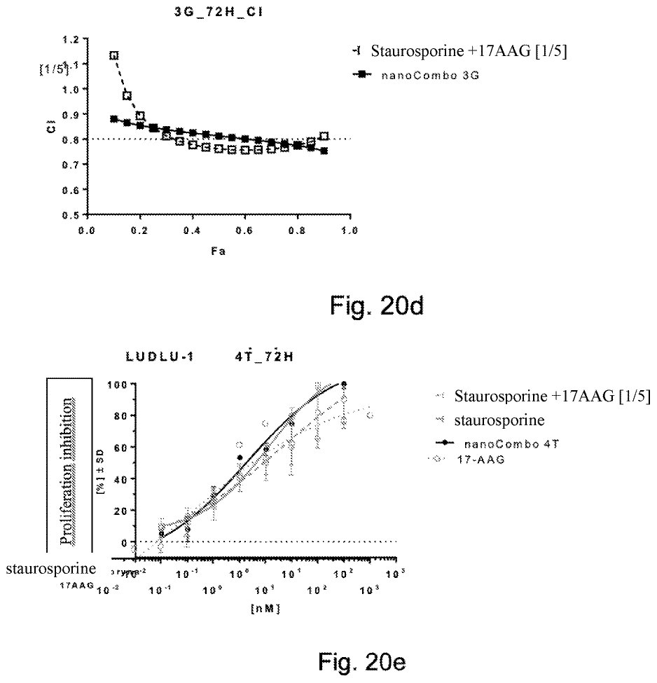

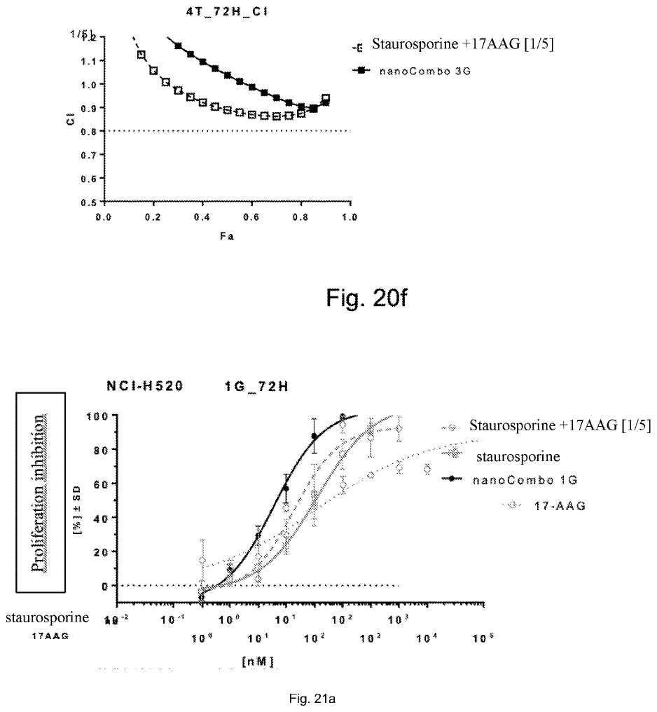

FIG. 20A, C, E—Growth kinetics of human lung cancer tumours NCI-H226 in mice treated with Staurosporine for different nanoparticles,

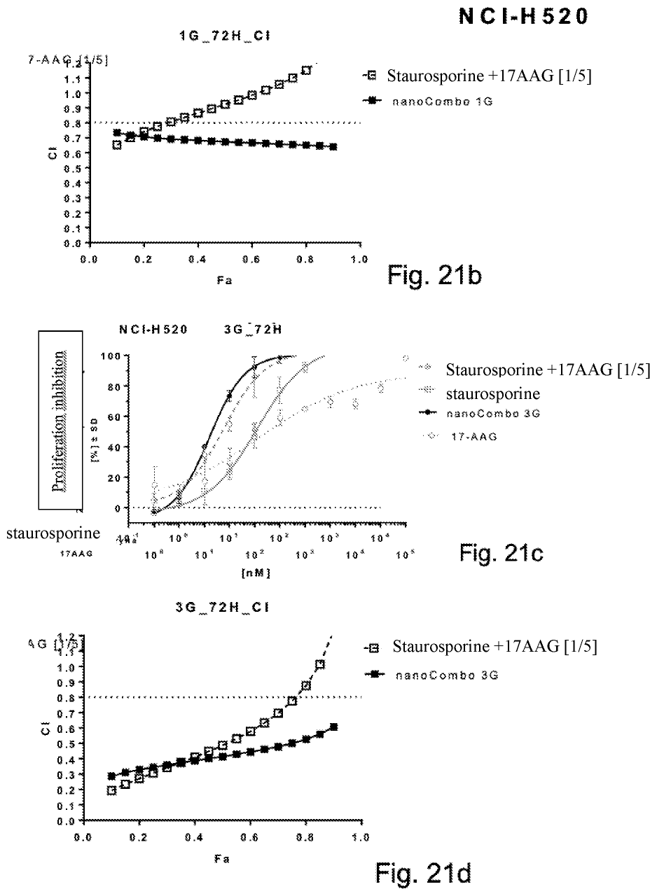

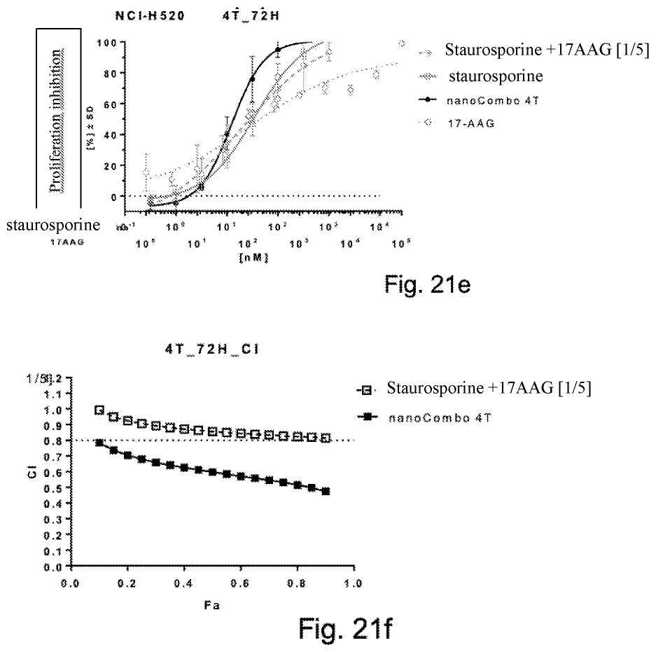

FIG. 21A, C, E—Growth kinetics of human lung cancer tumours LUDLU-1 in mice treated with Staurosporine for different nanoparticles,

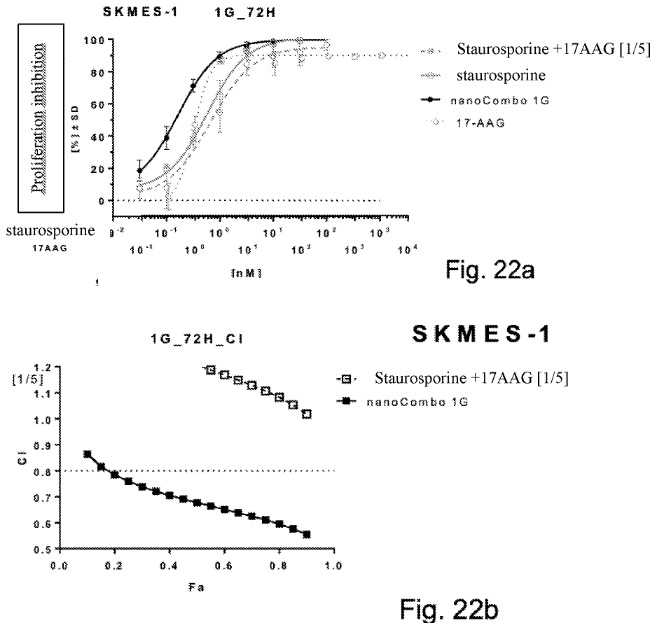

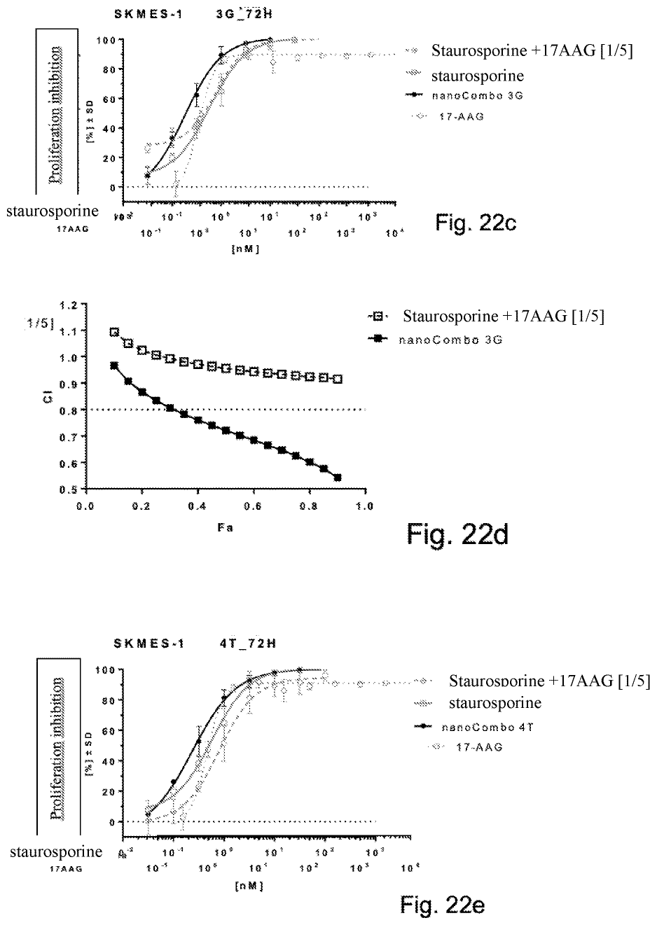

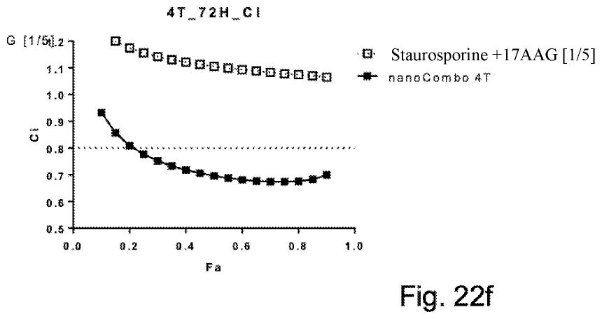

FIG. 22A, C, E—Growth kinetics of human lung cancer tumours NCI-H520 in mice treated with Staurosporine for different nanoparticles.

### EXAMPLE 1

The assay used 17-AAG and Staurosporine, which were dissolved in 100% DMSO and stored as 50 mM stock solutions at −80° C. Cell lines were used in the assay as shown in Table 1.

TABLE 1
List of cell lines detailing their origin

| Line symbol | Name of the line | Origin of the line |
|---|---|---|
| LUDLU-1 | Human lung squamous cell carcinoma | ECACC |
| SK-MES 1 | Human lung squamous cell carcinoma | ECACC |
| NCI-H520 | Human lung squamous cell carcinoma | ATCC |
| NCI-H226 | Human lung squamous cell carcinoma | ATCC |
| SK-MEL 3 | Human melanoma | ATCC |
| SW 1990 | Human pancreatic adenocarcinoma | ATCC |
| A-375 | Human melanoma | ATCC |
| BxPC-3 | Human pancreatic adenocarcinoma | ATCC |
| NCI-H1299 | Human non-small cell lung carcinoma | ATCC |
| NCI-H1975 | Human non-small cell lung carcinoma | ATCC |
| LN229 | Human glioblastoma | ATCC |

Cells were thawed from ampoules stored in liquid nitrogen and cultured in petri dishes in appropriate culture medium according to Table 2, in a humidified 5% CO₂ atmosphere at 37° C. Cells were passaged twice weekly using EDTA-Trypsin solution, pH 8. Counted cells with a viability of at least 90% as assessed with Trypan-Blue solution were used for anti-proliferative assays.

TABLE 2
List of cell lines and their corresponding culture medium

| Cell line | Growing medium |
|---|---|
| LUDLU-1 | RPMI-1640 (with glutamine), FBS 10% (v/v) |
| SK MES 1 | EBSS, glutamine 2 mM, 1% (v/v) non-essential amino acids |
| | (NEAA), FBS 10% (v/v) |
| NCI-H520 | RPMI-1640 + G.MAX, FBS 10% (v/v), 4.5 g/L glucose, |
| | 1 mM pyruvate |
| NCI-H226 | RPMI-1640 (with glutamine), FBS 10% (v/v) |
| BALB/3T3 | DMEM, FBS 10%, 2 mM glutamine |
| A-375 | DMEM, FBS 10%, 2 mM glutamine |
| SK-MEL 3 | McCoy 5A (with glutamine) FBS 10% |
| NCI- | RPMI-1640 (with glutamine;), FBS 10%, 2.5 g/L glucose, |
| H1299 | 1 mM pyruvate |
| NCI- | RPMI-1640 (with glutamine), FBS 10%, 2.5 g/L glucose, |
| H1975 | 1 mM pyruvate |
| BxPC-3 | RPMI-1640 + HEPES, FBS 10%, 2 mM glutamine |
| SW 1990 | RPMI-1640 (with glutamine) FBS 10% |

all culture media were supplemented with 100 U/mL penicillin, 100 μg/mL streptomycin

Twenty-four hours before the addition of the test compounds/combinations, cells were seeded into 384-well plates at 50 μL/well and a density of 10³ cells/well, in the appropriate culture medium. After incubation overnight, cells were treated with different concentrations of compounds or combinations of compounds mixed at different molar ratios, as described in the tables summarising the individual results, with a dilution factor of 3.16×.

Solutions of the compounds were diluted in the test medium-RPMI-1640 and Opti-MEM in a 1:1 ratio, and the medium was supplemented with 2 mM glutamine and 5% fetal bovine serum FBS. After 72 h, colourimetric assessment of cell growth was performed by SRB. Cells were fixed for 1 h with cold 25% (w/v) trichloroacetic acid (TCA) solution, washed five times with tap water, and stained with 0.4% (v/v) sulforhodamine B (SRB, solution in 1% (v/v) acetic acid) for 30 min. Unbound dye was removed by washing the plates (4×) in 1% (v/v) acetic acid, and protein-bound dye was extracted with 10 mM unbuffered Tris base and the optical density (A=540 nm) was determined in a BioTek Synergy H4 Hybrid computer microplate reader (BioTek Instruments USA). The entire washing/staining procedure was performed using a BioTek EL406 washing station (BioTek Instruments USA). The raw absorbance results were analysed in Microsoft Excel software using the formula:

$$\% ZahInh = ( ( \frac{{A}_{p} - {A}_{m}}{{A}_{k} - {A}_{m}} ) \times 100 ) - 100$$

Where:
% Zahlnh-inhibition of proliferation
Aₘ—absorbance of wells without cells (medium)
Aₖ—absorbance of control wells (cells treated with pure culture medium)
Aₚ—absorbance of wells treated with compounds

The proliferation inhibition data were then used to calculate the IC₅₀—the concentration of the test compound that inhibits the proliferation of 50% of the tumour cell population, calculated using the GraphPadPrism 7.0 non-linear model [Inhibitor] vs. response-Variable slope (four parameters).

Each compound at the indicated concentrations was tested in triplicate in one experiment. Each experiment was repeated three times. Data analysis was supplemented with synergy assessment using CalcuSyn software. The results of these analyses are given as combination index (CI) vs Fa (fraction affected-percentage of cells that responded to treatment with compounds/combinations). A CI value below 0.8 indicates synergism, 0.8-1.2 an additive effect, above 1.2 an antagonism.

TABLE 3a
Inhibition of LUDLU-1 line cell profiling for individual
concentrations of 17AAG, Staurosporine and their combinations at a ratio
of 100/1 including standard deviation
Cell line: LUDLU-1

| | | | | | | Staurosporine + 17AAG | | |
|---|---|---|---|---|---|---|---|---|
| 17AAG | | | Staurosporine | | | Ratio 1/100 | | |
| Concentration | | | Concentration | | | Concentration* | | |
| [nM] | Zah. | SD | [nM] | Zah. | SD | [nM] | Zah. | SD |
| 10000 | 86.3 | 3.1 | 100 | 84.5 | 5.7 | 100 | 98.7 | 0.8 |
| 3154.6 | 83.3 | 3.7 | 30 | 68.8 | 4.8 | 30 | 94.8 | 2.4 |
| 1000 | 78.6 | 4.1 | 10 | 53.6 | 8.4 | 10 | 86.4 | 3 |
| 313.9 | 74.1 | 3.8 | 3.1 | 50 | 3.6 | 3.1 | 76.9 | 2.3 |
| 100 | 66.9 | 6.1 | 1 | 31.7 | 8.9 | 1 | 72.7 | 3.8 |
| 31.4 | 47.8 | 8.5 | 0.31 | 13.1 | 2.1 | 0.31 | 51.9 | 10.4 |
| 10 | 20.4 | 6.5 | 0.1 | 3 | 8 | 0.1 | 25.5 | 11.7 |
| 3.1 | 6.1 | 7.3 | 0.0311 | 3.6 | 4.1 | 0.0311 | 10.8 | 6.5 |

*concentration refers to the concentration of staurosporine in the mixture of compounds, the concentration of 17AAG results from the molar ratio used

TABLE 3b
Values of affected fractions and corresponding combination index
obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| Cl | 0.95 | 0.9 | 0.86 | 0.82 | 0.8 | 0.77 | 0.75 | 0.73 | 0.71 | 0.69 | 0.68 | 0.66 | 0.64 | 0.62 | 0.59 | 0.57 | 0.53 |

TABLE 4A
Inhibition of cell prophylation of the SK-MES-1 line for individual
concentrations of 17AAG, Staurosporine and their combinations at a ratio
of 100/1 including standard deviation
Cell line: SK-MES-1

| | | | | | | Staurosporine + 17AAG | | |
|---|---|---|---|---|---|---|---|---|
| 17AAG | | | Staurosporine | | | Ratio 1/100 | | |
| Concentration | | | Concentration | | | Concentration* | | |
| [nM] | Zah. | SD | [nM] | Zah. | SD | [nM] | Zah. | SD |
| 10000 | 87 | 4 | 100 | 85.6 | 5.6 | 100 | 99.1 | 0.5 |
| 3139.2 | 86.9 | 5.8 | 30 | 83.6 | 5.7 | 30 | 97.7 | 0.5 |
| 1000 | 86.5 | 5.4 | 10 | 77 | 6.3 | 10 | 97.1 | 0.7 |
| 312.4 | 85.2 | 6.1 | 3.1 | 62.6 | 7.9 | 3.1 | 94.9 | 1 |
| 100 | 80 | 8.4 | 1 | 27.7 | 5.2 | 1 | 90.6 | 4 |
| 30 | 55.1 | 8.5 | 0.31 | 5 | 4.3 | 0.31 | 58.8 | 8.5 |
| 10 | 12.7 | 6.7 | 0.1 | −1.8 | 5.8 | 0.1 | 27.3 | 3.7 |
| 3.1 | 9.2 | 9.1 | 0.0311 | 1.9 | 4.2 | 0.0311 | 5.5 | 7 |

*concentration refers to the concentration of staurosporine in the mixture of compounds, the concentration of 17AAG results from the molar ratio used

TABLE 4b
Values of affected fractions and corresponding combination index
obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| Cl | 1.32 | 1.13 | 1.01 | 0.92 | 0.85 | 0.79 | 0.74 | 0.69 | 0.65 | 0.61 | 0.57 | 0.53 | 0.49 | 0.46 | 0.41 | 0.37 | 0.32 |

TABLE 5a
Inhibition of cell profiling of the NCI-H520 line for individual
concentrations of 17AAG, Staurosporine and their combinations at a ratio
of 1/1 including standard deviation
Cell line: NCI-H520

| | | | | | | Staurosporine + 17AAG | | |
|---|---|---|---|---|---|---|---|---|
| 17AAG | | | Staurosporine | | | Ratio 1/1 | | |
| Concentration | | | Concentration | | | Concentration* | | |
| [nM] | Zah. | SD | [nM] | Zah. | SD | [nM] | Zah. | SD |
| 10000 | 65.8 | 5.1 | 10000 | 97 | 1.7 | 10000 | 98.1 | 2.7 |
| 3139.2 | 63.5 | 4.6 | 3139.2 | 89.8 | 2.1 | 3139.2 | 97.7 | 2.7 |
| 1000 | 59.9 | 3.4 | 1000 | 88.1 | 2.7 | 1000 | 97.1 | 4.7 |
| 312.4 | 56.8 | 3.7 | 312.4 | 76.3 | 1.6 | 312.4 | 94.7 | 4.1 |
| 100 | 48.1 | 5.8 | 100 | 63.4 | 4.3 | 100 | 86.9 | 3 |
| 30 | 28.3 | 5.2 | 30 | 42.2 | 6.1 | 30 | 66.8 | 10.9 |
| 10 | 15.3 | 4 | 10 | 20.7 | 6.3 | 10 | 29.9 | 10.3 |
| 3.1 | 5.9 | 4.6 | 3.1 | 5.7 | 3.5 | 3.1 | 10.3 | 7.5 |

*concentration refers to the concentration of staurosporine in the mixture of compounds, the concentration of 17AAG results from the molar ratio used

TABLE 5b
Values of affected fractions and corresponding combination index
obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| Cl | 2.09 | 1.36 | 1.01 | 0.8 | 0.65 | 0.55 | 0.47 | 0.41 | 0.36 | 0.31 | 0.27 | 0.24 | 0.21 | 0.18 | 0.15 | 0.12 | 0.1 |

TABLE 6a
Inhibition of NCI-H226 line cell profiling for individual
concentrations of 17AAG, Staurosporine and their combinations at a ratio
of 1/1 including standard deviation
Cell line: NCI-H226

| | | | | | | Staurosporine + 17AAG | | |
|---|---|---|---|---|---|---|---|---|
| 17AAG | | | Staurosporine | | | Ratio 1/1 | | |
| Concentration | | | Concentration | | | Concentration* | | |
| [nM] | Zah. | SD | [nM] | Zah. | SD | [nM] | Zah. | SD |
| 10000 | 72.6 | 7 | 10000 | 97.7 | 1 | 10000 | 104.3 | 11.6 |
| 3139.2 | 71.9 | 9.5 | 3139.2 | 98.4 | 1.2 | 3139.2 | 101.5 | 4.8 |
| 1000 | 60.4 | 9.4 | 1000 | 93.8 | 2 | 1000 | 104.6 | 11 |
| 312.4 | 54.8 | 9.3 | 312.4 | 73.7 | 5.8 | 312.4 | 94.1 | 4.4 |
| 100 | 43.8 | 8.1 | 100 | 48 | 3.1 | 100 | 90.3 | 6.5 |
| 30 | 32 | 5.8 | 30 | 41.6 | 3.7 | 30 | 47.5 | 8.1 |
| 10 | 27.3 | 4.1 | 10 | 33.2 | 4 | 10 | 44.9 | 7.9 |
| 3.1 | 19.3 | 11 | 3.1 | 24.2 | 5 | 3.1 | 31.2 | 9.9 |

*concentration refers to the concentration of staurosporine in the mixture of compounds, the concentration of 17AAG results from the molar ratio used

TABLE 6b
Values of affected fractions and corresponding combination index
obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| Cl | 3.61 | 1.91 | 1.25 | 0.92 | 0.72 | 0.6 | 0.51 | 0.44 | 0.38 | 0.34 | 0.3 | 0.27 | 0.24 | 0.21 | 0.19 | 0.16 | 0.13 |

TABLE 7a
Inhibition of cell profiling of SK-MEL3 lines for individual
concentrations of 17AAG, Staurosporine and their combinations at a ratio
of 10/1 including standard deviation
Cell line: SK-MEL3

| | | | | | | Staurosporine + 17AAG | | |
|---|---|---|---|---|---|---|---|---|
| 17AAG | | | Staurosporine | | | Ratio 1/10 | | |
| Concentration | | | Concentration | | | Concentration* | | |
| [nM] | Zah. | SD | [nM] | Zah. | SD | [nM] | Zah. | SD |
| 10000 | 76.6 | 2.3 | 1000 | 84.5 | 4.6 | 10000 | 92.6 | 11.2 |
| 3154.6 | 60.7 | 3.5 | 315.5 | 64.8 | 3.9 | 3139.2 | 83.8 | 12.3 |
| 1000 | 58.5 | 2.6 | 100 | 57.2 | 4 | 1000 | 63.1 | 5.5 |
| 315.5 | 58.1 | 2.7 | 31.4 | 45 | 10.6 | 312.4 | 60.1 | 3.9 |
| 100 | 48.5 | 3.5 | 10 | 27.5 | 5.7 | 100 | 53.3 | 4 |
| 31.4 | 40.3 | 1.5 | 3.1 | 17.6 | 6.3 | 30 | 37.3 | 3.9 |
| 10 | 14 | 8.5 | 1 | 9.9 | 5.7 | 10 | 22.3 | 6.8 |
| 3.1 | 1.1 | 6.8 | 0.3109 | 9 | 6.8 | 3.1 | 12.8 | 5 |

*concentration refers to the concentration of staurosporine in the mixture of compounds, the concentration of 17AAG results from the molar ratio used

TABLE 7b
Values of affected fractions and corresponding combination index
obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| Cl | 0.54 | 0.64 | 0.74 | 0.82 | 0.91 | 0.99 | 1.08 | 1.17 | 1.27 | 1.37 | 1.49 | 1.62 | 1.77 | 1.96 | 2.19 | 2.52 | 3.04 |

TABLE 8a
Inhibition of cell prophylation of SW1990 for individual
concentrations of 17AAG, Staurosporine and their combinations at
100/1 including standard deviation
Mobile line: SW1990

| | | | | | | Staurosporine + 17AAG | | |
|---|---|---|---|---|---|---|---|---|
| 17AAG | | | Staurosporine | | | Ratio 1/100 | | |
| Concentration | | | Concentration | | | Concentration* | | |
| [nM] | Zah. | SD | [nM] | Zah. | SD | [nM] | Zah. | SD |
| 10000 | 92.6 | 2.7 | 100 | 73.4 | 2.1 | 100 | 86.2 | 3.2 |
| 3154.6 | 93.5 | 2.3 | 31.4 | 70 | 1.8 | 31.4 | 94.3 | 3.1 |
| 1000 | 92.9 | 2.4 | 10 | 64 | 3.8 | 10 | 98.9 | 2 |
| 315.5 | 90.6 | 1.9 | 3.1 | 49.3 | 4.3 | 3.1 | 96.6 | 1.6 |
| 100 | 81.7 | 2.3 | 1 | 34.8 | 3 | 1 | 82.8 | 3.9 |
| 31.4 | 40.7 | 9.6 | 0.3109 | 18.7 | 8.3 | 0.3109 | 41.9 | 6.2 |
| 10 | 5.7 | 6.9 | 0.0985 | 13.3 | 6 | 0.0985 | 11.6 | 6.9 |
| 3.1 | −0.2 | 7.7 | 0.0311 | 10.5 | 3.2 | 0.0311 | 2.2 | 6.7 |

*concentration refers to the concentration of staurosporine in the mixture of compounds, the concentration of 17AAG results from the molar ratio used

TABLE 8b
Values of affected fractions and corresponding combination index
obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| Cl | 3.46 | 2.02 | 1.47 | 1.19 | 1.04 | 0.94 | 0.88 | 0.83 | 0.8 | 0.77 | 0.76 | 0.74 | 0.73 | 0.71 | 0.7 | 0.69 | 0.68 |

TABLE 9a
Inhibition of A375 cell profiling for individual concentrations of
17AAG. Staurosporine and their combinations at 100/1 including standard deviation
Mobile line: A375

| 17AAG | | | Staurosporine | | | Staurosporine + 17AAG | | |
|---|---|---|---|---|---|---|---|---|
| Concentration | | | Concentration | | | Ratio 1/100 | | |
| [nM] | Zah. | SD | [nM] | Zah. | SD | Concentration* [nM] | Zah. | SD |
| 10000 | 97 | 0.6 | 100 | 82.1 | 2 | 100 | 97.6 | 0.8 |
| 3154.6 | 97.1 | 0.9 | 31.4 | 57.8 | 2.9 | 31.4 | 94.9 | 1.3 |
| 1000 | 96.3 | 1.1 | 10 | 36.8 | 4.6 | 10 | 95 | 0.7 |
| 315.5 | 91.6 | 1.5 | 3.1 | 23.4 | 4.3 | 3.1 | 93.5 | 1.4 |
| 100 | 89.2 | 1.5 | 1 | 16.7 | 4.9 | 1 | 89.6 | 2 |
| 31.4 | 83.2 | 3.4 | 0.3109 | −3 | 4.2 | 0.3109 | 79.7 | 4.4 |
| 10 | 53.3 | 7 | 0.0985 | −9 | 3.3 | 0.0985 | 61.8 | 4.9 |
| 3.1 | 24.1 | 9.9 | 0.0311 | −9.7 | 1.9 | 0.0311 | 32.5 | 3.7 |

*concentration refers to the concentration of staurosporine in the mixture of compounds. the concentration of 17AAG results from the molar ratio used

TABLE 9b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| Cl | 0.19 | 0.23 | 0.27 | 0.31 | 0.34 | 0.38 | 0.41 | 0.45 | 0.49 | 0.53 | 0.58 | 0.63 | 0.7 | 0.78 | 0.88 | 1.01 | 1.23 |

TABLE 10a
Inhibition of BxPC3 line cell profiling for individual concentrations
of 17AAG. Staurosporine and their combinations at a ratio of 100/1 including standard deviation
Cell line: BxPC3

| 17AAG | | | Staurosporine | | | Staurosporine + 17AAG | | |
|---|---|---|---|---|---|---|---|---|
| Concentration | | | Concentration | | | Ratio 1/100 | | |
| [nM] | Zah. | SD | [nM] | Zah. | SD | Concentration* [nM] | Zah. | SD |
| 10000 | 95.2 | 2.1 | 100 | 73.9 | 6.1 | 100 | 97.7 | 1.6 |
| 3154.6 | 95.3 | 2.3 | 31.4 | 52.4 | 9.6 | 31.4 | 95.2 | 2.1 |
| 1000 | 89.4 | 5.5 | 10 | 35.3 | 8.4 | 10 | 95.1 | 3.7 |
| 315.5 | 75.9 | 10.6 | 3.1 | 21.9 | 4.5 | 3.1 | 86.1 | 5.9 |
| 100 | 54.7 | 10.7 | 1 | 6.4 | 7.2 | 1 | 66 | 12.5 |
| 31.4 | 24.4 | 8 | 0.3109 | −0.4 | 0.3 | 0.3109 | 31.4 | 8.4 |
| 10 | 2.7 | 1.6 | 0.0985 | −0.5 | 2.4 | 0.0985 | 19.2 | 6.6 |
| 3.1 | 1.7 | 2.3 | 0.0311 | −1.9 | 1.1 | 0.0311 | 6.3 | 2.8 |

*concentration refers to the concentration of staurosporine in the mixture of compounds. the concentration of 17AAG results from the molar ratio used

TABLE 10b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| Cl | 0.31 | 0.33 | 0.35 | 0.37 | 0.38 | 0.4 | 0.41 | 0.42 | 0.44 | 0.46 | 0.47 | 0.49 | 0.51 | 0.53 | 0.56 | 0.6 | 0.65 |

TABLE 11a
Inhibition of cell profiling of NCI-H1299 lines for individual concentrations of 17AAG.
Staurosporine and their combinations at 100/1 including standard deviation
Cell line: NCI-H1299

| 17AAG | | | Staurosporine | | | Staurosporine + 17AAG | | |
|---|---|---|---|---|---|---|---|---|
| Concentration | | | Concentration | | | Ratio 1/100 | | |
| [nM] | Zah. | SD | [nM] | Zah. | SD | Concentration* [nM] | Zah. | SD |
| 10000 | 95.3 | 1 | 100 | 86.1 | 1.4 | 100 | 97.7 | 0.9 |
| 3154.6 | 96.8 | 0.3 | 31.4 | 83.5 | 2.9 | 31.4 | 95.2 | 0.7 |
| 1000 | 96.9 | 0.6 | 10 | 77.5 | 2.6 | 10 | 95.1 | 0.3 |
| 315.5 | 93.4 | 1.7 | 3.1 | 66.2 | 5.9 | 3.1 | 86.1 | 0.7 |
| 100 | 80.8 | 10.2 | 1 | 34.5 | 6.3 | 1 | 66 | 4.4 |
| 31.4 | 43.4 | 11.5 | 0.3109 | 8.4 | 5.1 | 0.3109 | 31.4 | 2.9 |
| 10 | 10.5 | 9.7 | 0.0985 | 2.8 | 2.2 | 0.0985 | 19.2 | 5.6 |
| 3.1 | 5.1 | 4.5 | 0.0311 | −0.8 | 4.1 | 0.0311 | 6.3 | 6.5 |

*concentration refers to the concentration of staurosporine in the mixture of compounds. the concentration of 17AAG results from the molar ratio used

TABLE 11b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| Cl | 0.89 | 0.85 | 0.82 | 0.81 | 0.79 | 0.78 | 0.77 | 0.76 | 0.76 | 0.75 | 0.74 | 0.74 | 0.73 | 0.73 | 0.72 | 0.72 | 0.72 |

TABLE 12a
Inhibition of cell prophylation of line HCI-H1975 for individual concentrations of 17AAG.
Staurosporine and their combinations at 100/1 including standard deviation
Cell line: NCI-H1975

| 17AAG | | | Staurosporine | | | Staurosporine + 17AAG | | |
|---|---|---|---|---|---|---|---|---|
| Concentration [nM] | Zah. | SD | Concentration [nM] | Zah. | SD | Concentration* [nM] | Zah. | SD |
| 10000 | 86.6 | 1.7 | 100 | 82.5 | 2.7 | 100 | 98.9 | 0.4 |
| 3154.6 | 88.4 | 1.5 | 31.4 | 67.6 | 2.2 | 31.4 | 98.7 | 0.7 |
| 1000 | 88.5 | 2.3 | 10 | 61.6 | 2.3 | 10 | 98.1 | 0.6 |
| 315.5 | 87.6 | 2.1 | 3.1 | 44.2 | 4.9 | 3.1 | 95.9 | 0.7 |
| 100 | 84.1 | 3.1 | 1 | 16.1 | 3.6 | 1 | 88.3 | 2.8 |
| 31.4 | 66.7 | 7.8 | 0.3109 | 4.6 | 2.5 | 0.3109 | 66.7 | 7.2 |
| 10 | 25.2 | 8 | 0.0985 | 0.8 | 1.5 | 0.0985 | 33.3 | 8.8 |
| 3.1 | 10 | 4.4 | 0.0311 | −3 | 3 | 0.0311 | 4.6 | 2.6 |

*-concentration refers to the concentration of staurosporine in the mixture of compounds. the concentration of 17AAG results from the molar ratio used

TABLE 12b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| Cl | 1.81 | 1.56 | 1.4 | 1.28 | 1.18 | 1.1 | 1.04 | 0.97 | 0.92 | 0.86 | 0.81 | 0.77 | 0.72 | 0.67 | 0.61 | 0.55 | 0.49 |

TABLE 13a
Inhibition of LN229 line cell profiling for individual concentrations of 17AAG. Staurosporine
and their combinations at a ratio of 5/1 including standard deviation
Mobile line: LN229

| | | | | | | Staurosporine + 17AAG | | |
|---|---|---|---|---|---|---|---|---|
| 17AAG | | | Staurosporine | | | Ratio 1/5 | | |
| Concentration [nM] | Zah. | SD | Concentration [nM] | Zah. | SD | Concentration* [nM] | Zah. | SD |
| 10000.5 | 81.5 | 1.2 | 1000 | 93.4 | 4.1 | 1000 | 98.9 | 3.9 |
| 3162.5 | 71.6 | 6.8 | 316.3 | 81.4 | 5.7 | 316.3 | 98.7 | 10 |
| 1000 | 62.9 | 11.2 | 100 | 70.2 | 5 | 100 | 98.1 | 4.7 |
| 316.3 | 38.7 | 11.1 | 31.6 | 60.4 | 4 | 31.6 | 95.9 | 4.5 |
| 100 | 28.9 | 10.7 | 10 | 49.8 | 9.7 | 10 | 88.3 | 2.9 |
| 31.6 | 18.4 | 18.9 | 3.2 | 35.8 | 4.6 | 3.2 | 66.7 | 4.2 |
| 10 | 3.1 | 4.2 | 1 | 33.7 | 2.3 | 1 | 33.3 | 0.9 |
| 3.2 | 3.3 | 4.1 | 0.3163 | 31.1 | 0 | 0.3163 | 4.6 | 3.8 |

*concentration refers to the concentration of staurosporine in the mixture of compounds. the concentration of 17AAG results from the molar ratio used

TABLE 13b
Values of affected fractions and corresponding combination ndex obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| Cl | 0.99 | 0.89 | 0.83 | 0.78 | 0.75 | 0.72 | 0.7 | 0.69 | 0.68 | 0.67 | 0.67 | 0.67 | 0.67 | 0.69 | 0.71 | 0.75 | 0.84 |

### EXAMPLE 2

Synthesis of nanoCombo Nanoparticles

200 mg of PVA (Mw 30 000-70 000 g/mol), 80 mg of PLGA (50:50 copolymer Mw 24 000-38 000 g/mol) and 17AAG and Staurosporine were weighed according to Table 14. PVA was dissolved in 10 ml of PBS buffer pH 7.4 using ultrasound until the PVA crystals were completely dissolved, then left in the refrigerator overnight. PLGA, Staurosporine and 17AAG were dissolved in 5 ml of dichloromethane. This was cooled to 4° C. The dichloromethane solution with PLGA, Staurosporine and 17AAG was then added to a large glass vessel (50 ml bottle) followed by the PVA aqueous solution, again chilled in the fridge for 30 minutes. After this time, the whole mixture was stirred until an emulsion was obtained using an ultrasonic probe for 1 minute, amplitude 80%. The ultrasonic probe was immersed at the boundary between the two phases about 5 mm above the phase boundary.

After sonification, a homogeneous emulsion was obtained. The emulsion was poured into a 50 ml crystalliser with a mixing element making sure that all the emulsion was transferred into the crystalliser. The bottle was washed with 2 ml of PBS 7.4 (the solution was placed in the crystalliser after washing). The whole was left on a magnetic stirrer for 4 h (400 RPM, 37° C.) until the dichloromethane was completely evaporated. The solution was then made up to 15 mL with PBS buffer pH7.4. PLGA/PVA nanoparticles loaded with Staurosporine and 17AAG were added to a glass bottle with a stirring element followed by 200 μL of liposomes (DPPC/Cholesterol/DSPE_PEG(2000)NH2 (1/0.5/0.04) 10 mg/ml) synthesised in 0.9% NaCl for every 1 mL of polymeric nanoparticles. The sample was heated in a water bath for 2 h, 60° C., stirring with a magnetic stirrer with heating (400 RPM). It was then left in the refrigerator for 16 h. Subsequently, the % EE encapsulation factor was determined by chromatography on a Sephadex G-25 packed column (described below).

The rest of the nanoparticles were purified using a high-speed centrifuge (15,000 RPM, 4 min), washing twice with PBS 7.4 buffer. After each addition of PBS 7.4, the nanoparticles were redispersed using ultrasound for 5 seconds. After the last centrifugation, non-ionic surfactant (40% solution) was added at a volume ratio of 0.02:1 (surfactant: nanoparticles) or glucose until a concentration of 2 mg/ml was reached (according to Table 1). The nanoparticles were then sterilised using sterile PTFE-filled hydrophilic syringe filters with a pore diameter of 0.45 μm and size and PDI were determined. The data are shown in FIGS. 12-14.

The concentration of loaded drugs and % EE were determined using:
1 UV-Vis spectroscopy against a calibration curve at 372 nm and 333 nm using the principle of absorbance additivity. The nanoparticle sample was dissolved in DMSO with hydrochloric acid according to the ratio: 50 μL of sample, 50 μL of 0.3M HCl, 50 μL of PBS 7.4 and 850 μL of DMSO and a spectrum was taken.
2. chromatographic method, purifying 0.5 ml sample of final nanoparticles Purification was performed on Sephadex G-25 columns with drug separation. After depositing a 0.5 ml sample of unpurified nanoparticles on the column, 8 fractions of 1 ml were collected with PBS buffer pH 7.4. The content of each fraction was controlled by UV-Vis spectroscopy. Fractions 4-7 containing nanoparticles were pooled together and drug concentrations on the nanoparticles were determined by UV-Vis spectroscopy against a calibration curve using the principle of additivity of absorbance by dissolving the sample from fractions 4-7 in DMSO with hydrochloric acid according to the ratio: 50 μL of sample, 50 μL of 0.3M HCl, 50 μL of PBS 7.4 and 850 μL of DMSO. Subsequently, unbound 17AAG from the column was eluted from the column by washing the chromatography column with 25 ml of PBS buffer pH 7.4 and the concentration of unbound 17AAG relative to the 17AAG calibration curve in PBS buffer at 333 nm was determined. Unbound Staurosporine was then eluted from the column by washing the column with 25 ml of Ethanol:Water solution (1:1). The concentration of free Staurosporine was determined by UV-Vis spectroscopy against a calibration curve at 372, 294 and 353 nm.

The post-column concentration of the drugs in the liposomes is summarised in Table 14.

TABLE 14
nanoCombo nanoparticle synthesis parameters with values for
drug encapsulation rates and drug concentrations in the final nanoparticles.

| | 17AAG | | Addition of a | %EE | | | | |
|---|---|---|---|---|---|---|---|---|
| | [mg]. | ST [mg] | stabilising | 17AAG | % EE ST | C17AAG | | C17AAG/ |
| Code NPs | weight | weight | agent | [%] | [%] | [g/L]. | CST [g/L] | CST |
| nanoCombo 1G | 8.4 | 1.7 | Glucose | 53.4 | 61.5 | 0.0356 | 0.0082 | 4.3 |
| nanoCombo 3G | 10.0 | 1.0 | Glucose | 46.2 | 63 | 0.0308 | 0.0042 | 7.3 |
| nanoCombo 4T | 5.0 | 1.0 | Tween20 | 60.2 | 75 | 0.0401 | 0.0100 | 4 |

### EXAMPLE III

The assay used 17-AAG/Tanespimycin and Staurosporine, and the nanoparticles nanoCombo 1G, nanoCombo 3G, nanoCombo 4T, nanoStauro G, nanoStauro T, nanoEmpty G, nanoEmpty T which were dissolved in 100% DMSO and stored as 50 mM stock solutions at −80° C.

Cell lines were used in the assay as shown in Table 15.

TABLE 15
List of cell lines by origin

| Mobile line | | |
|---|---|---|
| symbol | Name of cell line | Origin of the line |
| LUDLU-1 | Human lung | ECACC |
| | squamous cell | |
| | carcinoma | |
| SK-MES 1 | Human lung | ECACC |
| | squamous cell | |
| | carcinoma | |
| NCI-H520 | Human lung | ATCC |
| | squamous cell | |
| | carcinoma | |
| NCI-H226 | Human lung | ATCC |
| | squamous cell | |
| | carcinoma | |
| A-172 | Human brain | ATCC |
| | glioblastoma | |
| Hs683 | Human brain glioma | ATCC |
| 8MBGA | Human brain glioma | Provided by Medical University of |
| LN-229 | Human brain glioma | Lublin (Anna Boguszewska- |
| | | Czubara) |

Cells were thawed from ampoules stored in liquid nitrogen and cultured in petri dishes in the appropriate culture medium according to Table 16, in a humidified 5% CO₂ atmosphere at 37° C. Cells were passaged twice weekly using EDTA-Trypsin solution, pH 8, as a separating agent. Counted cells with a viability of at least 90% as assessed with Trypan-Blue solution were used for anti-proliferation assays.

TABLE 16
List of cell lines with corresponding culture medium

| Cell line | Growing medium |
|---|---|
| LUDLU-1 | RPMI-1640 (with glutamine) FBS 10% (v/v) |
| SK MES | EBSS, glutamine 2 mM, 1% (v/v) non-essential amino acids |
| 1 | (NEAA), FBS 10% (v/v) |
| NCI- | RPMI-1640 + G.MAX, FBS 10% (v/v), 4.5 g/L glucose, 1 mM |
| H520 | pyruvate |
| NCI- | RPMI-1640 (with glutamine), FBS 10% (v/v) |
| H226 | |
| A-172 | DMEM, FBS 10%, 2 mM glutamine |
| Hs683 | DMEM, FBS 10%, 2 mM glutamine |
| 8MBGA | EBSS, glutamine 2mM, 1% (v/v) non-essential amino acids |
| | (NEAA), FBS 10% (v/V) |
| LN-229 | DMEM, FBS 10%, 2 mM glutamine |

all culture media were supplemented with 100 U/mL penicillin, 100 μg/mL streptomycin

Twenty-four hours before the addition of the test compounds/combinations, cells were seeded into 384-well plates at 50 μL/well and a density of 10³ cells/well, in the appropriate culture medium. After incubation overnight, cells were treated with different concentrations of compounds or combinations of compounds mixed at different molar ratios, as described in the respective tables summarising the individual results, with a dilution factor of 3.16×. Solutions of the compounds were diluted in the test medium—RPMI-1640 and Opti-MEM in a 1:1 ratio, and the medium was supplemented with 2 mM glutamine and 5% fetal bovine serum FBS.

After 72 h, colourimetric assessment of cell growth was performed by SRB. Cells were fixed for 1 h with cold 25% (w/v) trichloroacetic acid (TCA) solution, washed five times with tap water, and stained with 0.4% (v/v) sulforhodamine B (SRB, solution in 1% (v/v) acetic acid) for 30 min. Unbound dye was removed by washing the plates (4×) in 1% (v/v) acetic acid, and protein-bound dye was extracted with 10 mM unbuffered Tris base and the optical density (A=540 nm) was determined in a BioTek Synergy H4 Hybrid computer microplate reader (BioTek Instruments USA). The entire washing/staining procedure was performed using a BioTek EL406 washing station (BioTek Instruments USA). The raw absorbance results were analysed in Microsoft Excel software using the formula:

$$\% ZahInh = ( ( \frac{{A}_{p} - {A}_{m}}{{A}_{k} - {A}_{m}} ) \times 100 ) - 100$$

Where:
% Zahlnh-inhibition of proliferation
Aₘ—absorbance of wells without cells (medium)
Aₖ—absorbance of control wells (cells treated with pure culture medium)
Aₚ—absorbance of wells treated with compounds

The proliferation inhibition data were then used to calculate the IC₅₀—the concentration of the test compound that inhibits the proliferation of 50% of the tumour cell population, calculated using the GraphPadPrism 7.0 non-linear model [Inhibitor] vs. response-Variable slope (four parameters).

Each compound at the indicated concentrations was tested in triplicate in one experiment. Each experiment was repeated three times. Data analysis was supplemented with synergy assessment using CalcuSyn software. The results of these analyses are given as combination index (CI) vs Fa (percentage of cells that responded to treatment with compounds/combinations). A CI value below 0.8 indicates synergism, 0.8-1.2 an additive effect, above 1.2 an antagonism.

TABLE 17a
Inhibition of cell profiling of line 8MGBA for individual concentrations of 17AAG. Staurosporine
and their combinations at a ratio of 5/1 including standard deviation
Mobile line: 8MGBA

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 1G]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 1000 | 91.9 | 0.6 | 100 | 97.9 | 1.5 | 100 | 90.3 | 7.7 | 100 | 101. 8 | 2.4 |
| 316.3 | 86 | 4.1 | 30 | 96.2 | 1 | 30 | 88 | 7 | 30 | 98.2 | 2.7 |
| 100 | 84.3 | 7.4 | 10 | 86.7 | 8.1 | 10 | 76.8 | 7.3 | 10 | 94 | 4.2 |
| 31.6 | 88.5 | 4.1 | 3.1 | 44.8 | 5.1 | 3.1 | 36 | 8 | 3.1 | 77.7 | 9 |
| 10 | 89.5 | 0.5 | 1 | 32.6 | 13.8 | 1 | 2.6 | 3.5 | 1 | 48.3 | 10.6 |
| 3.2 | 64.9 | 7.6 | 0.31 | 4.3 | 0.1 | 0.31 | −2.4 | 8.5 | 0.31 | 26.9 | 4 |
| 1 | 9.8 | 9 | 0.1 | 0.6 | 1.2 | 0.1 | 4.4 | 5.1 | 0.1 | 6.5 | 2 |
| 0.3163 | −3.9 | 11.2 | 0.0311 | 0.3 | 8.3 | 0.0311 | −5.1 | 1 | 0.0311 | 3.9 | 4.7 |

*concentration refers to the concentration of staurosporine in the mixture of compounds. the concentration of 17AAG results from the molar ratio used

TABLE 17b
Values of affected fractions and corresponding combination
index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| Cl | 1.8 | 1.66 | 1.6 | 1.55 | 1.51 | 1.48 | 1.44 | 1.42 | 1.39 | 1.36 | 1.34 | 1.31 | 1.29 | 1.27 | 1.25 | 1.25 | 1.27 |
| CZAST | | | | | | | | | | | | | | | | | |
| Cl | 0.5 | 0.52 | 0.49 | 0.48 | 0.46 | 0.45 | 0.44 | 0.43 | 0.42 | 0.41 | 0.4 | 0.39 | 0.38 | 0.37 | 0.36 | 0.35 | 0.33 |
| Nano | | | | | | | | | | | | | | | | | |

TABLE 18a
Inhibition of cell profiling of line 8MGBA for individual concentrations of 17AAG. Staurosporine
and their combinations at a ratio of 5/1 including standard deviation
Mobile line: 8MGBA

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 3G]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 1000 | 91.9 | 0.6 | 100 | 97.9 | 1.5 | 100 | 98.1 | 1.6 | 100 | 101 | 2.2 |
| 316.3 | 86 | 4.1 | 30 | 96.2 | 1 | 30 | 98.9 | 2.3 | 30 | 98.5 | 1.2 |
| 100 | 84.3 | 7.4 | 10 | 86.7 | 8.1 | 10 | 89.1 | 2.5 | 10 | 95.5 | 3.4 |
| 31.6 | 88.5 | 4.1 | 3.1 | 44.8 | 5.1 | 3.1 | 56 | 0.4 | 3.1 | 82.9 | 7.4 |
| 10 | 89.5 | 0.5 | 1 | 24.6 | 1.6 | 1 | 25.9 | 3 | 1 | 49.4 | 13.7 |
| 3.2 | 64.9 | 7.6 | 0.31 | 4.3 | 0.1 | 0.31 | 13.2 | 0.9 | 0.31 | 27.3 | 8 |
| 1 | 9.8 | 9 | 0.1 | 0.6 | 1.2 | 0.1 | 6.1 | 3.9 | 0.1 | 6.6 | 0.8 |
| 0.3163 | −3.9 | 11.2 | 0.0311 | −4.3 | 3 | 0.0311 | 11.6 | 4.1 | 0.0311 | 1 | 4.1 |

*concentration refers to the concentration of staurosporine in the mixture of compounds. the concentration of 17AAG results from the molar ratio used

TABLE 18b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 1.5 | 1.26 | 1.1 | 1 | 0.92 | 0.86 | 0.82 | 0.79 | 0.76 | 0.75 | 0.75 | 0.76 | 0.79 | 0.85 | 0.96 | 1.15 | 1.58 |
| CZAST | | | | | | | | | | | | | | | | | |
| CI | 0.5 | 0.52 | 0.49 | 0.48 | 0.46 | 0.45 | 0.44 | 0.43 | 0.42 | 0.41 | 0.4 | 0.39 | 0.38 | 0.37 | 0.36 | 0.35 | 0.33 |
| Nano | | | | | | | | | | | | | | | | | |

TABLE 19a
Inhibition of cell profiling of line 8MGBA for individual concentrations of 17AAG,
Staurosporine and their combinations at a ratio of 5/1 including standard deviation
Cell line: 8MGBA

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 4T]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 1000 | 91.9 | 0.6 | 100 | 90.7 | 18.6 | 100 | 90.2 | 18.5 | 100 | 99.7 | 2.8 |
| 316.3 | 86 | 4.1 | 30 | 83.7 | 26.4 | 30 | 81.8 | 16.8 | 30 | 100.2 | 1.3 |
| 100 | 84.3 | 7.4 | 10 | 69.7 | 16.7 | 10 | 62.3 | 13.6 | 10 | 74.6 | 0.4 |
| 31.6 | 88.5 | 4.1 | 3.1 | 58.9 | 14 | 3.1 | 49.8 | 11 | 3.1 | 58.4 | 3.9 |
| 10 | 89.5 | 0.5 | 1 | 40.6 | 8.7 | 1 | 41.8 | 6.1 | 1 | 53.2 | 0.6 |
| 3.2 | 64.9 | 7.6 | 0.31 | 28.1 | 6.1 | 0.31 | 23 | 9.3 | 0.31 | 33.6 | 8.1 |
| 1 | 9.8 | 9 | 0.1 | 22.3 | 5 | 0.1 | 16.3 | 1.5 | 0.1 | 7.8 | 7.8 |
| 0.3163 | -3.9 | 11.2 | 0.0311 | 12.1 | 4 | 0.0311 | 8.8 | 6 | 0.0311 | 5.2 | 2.1 |

*concentration refers to the concentration of staurosporine in the mixture of compounds, the concentration of 17AAG results from the molar ratio used

TABLE 19b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 0.5 | 0.52 | 0.49 | 0.48 | 0.46 | 0.45 | 0.44 | 0.43 | 0.42 | 0.41 | 0.4 | 0.39 | 0.38 | 0.37 | 0.36 | 0.35 | 0.33 |
| CZAST | | | | | | | | | | | | | | | | | |
| CI | 1.4 | 1.28 | 1.22 | 1.18 | 1.15 | 1.11 | 1.09 | 1.06 | 1.03 | 1.01 | 0.99 | 0.96 | 0.94 | 0.91 | 0.88 | 0.85 | 0.81 |
| Nano | | | | | | | | | | | | | | | | | |

TABLE 20a
Inhibition of cell profiling of A172 lines for individual concentrations of 17AAG,
Staurosporine and their combinations at a ratio of 5/1 including standard deviation
Mobile line: A172

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 1G]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 1000 | 81.1 | 9.8 | 100 | 97.9 | 2.6 | 100 | 82.4 | 12.1 | 100 | 96.8 | 0.5 |
| 316.3 | 64.6 | 7.1 | 30 | 96.2 | 7.8 | 30 | 63.9 | 12.8 | 30 | 93.5 | 5.1 |
| 100 | 37.4 | 4.2 | 10 | 86.7 | 6.2 | 10 | 58.9 | 5.1 | 10 | 94.6 | 0.8 |
| 31.6 | 20.1 | 6 | 3.1 | 44.8 | 7.9 | 3.1 | 32.3 | 12.9 | 3.1 | 81.3 | 5.6 |
| 10 | 7.8 | 5.9 | 1 | 32.6 | 13.2 | 1 | 17.5 | 2.5 | 1 | 65.7 | 6.8 |
| 3.2 | 4.7 | 5.5 | 0.31 | 4.3 | 5.3 | 0.31 | 8.4 | 6.4 | 0.31 | 37.8 | 5 |
| 1 | 15.7 | 3.7 | 0.1 | 0.6 | 1.5 | 0.1 | 6.2 | 13 | 0.1 | 16.6 | 3.7 |
| 0.3163 | 6 | 5.2 | 0.0311 | 0.3 | 3.3 | 0.0311 | 5 | 2.4 | 0.0311 | 4.4 | 1.2 |

*concentration refers to the concentration of staurosporine in the mixture of compounds, the concentration of 17AAG results from the molar ratio used

TABLE 20b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 1.6 | 1.57 | 1.55 | 0 | 0 | 0 | 0 | 0 | 0 | 0 | 0 | 0 | 0 | 0 | 0 | 0 | 0 |
| CZAST | | | | | | | | | | | | | | | | | |
| CI | 0.9 | 0.9 | 0.92 | 0.93 | 0.94 | 0.95 | 0.97 | 0.98 | 0.99 | 1 | 1.01 | 1.03 | 1.04 | 1.06 | 1.08 | 1.1 | 1.14 |
| Nano | | | | | | | | | | | | | | | | | |

TABLE 21a
Inhibition of cell profiling of line 8MGBA for individual concentrations of 17AAG,
Staurosporine and their combinations at a ratio of 5/1 including standard deviation
Mobile line: 8MGBA

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 3G]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 1000 | 81.1 | 9.8 | 100 | 94.5 | 2.6 | 100 | 94 | 4.6 | 100 | 95.6 | 1 |
| 316.3 | 64.6 | 7.1 | 30 | 92.4 | 7.8 | 30 | 94.4 | 6.7 | 30 | 95.6 | 2.4 |
| 100 | 37.4 | 4.2 | 10 | 92.2 | 6.2 | 10 | 94.1 | 0.5 | 10 | 95.2 | 3.1 |
| 31.6 | 20.1 | 6 | 3.1 | 76.8 | 7.9 | 3.1 | 78.4 | 5.8 | 3.1 | 90.3 | 5.1 |
| 10 | 7.8 | 5.9 | 1 | 32.5 | 7.9 | 1 | 54.7 | 5.1 | 1 | 58.6 | 7.2 |
| 3.2 | 4.7 | 5.5 | 0.31 | 13.9 | 5.3 | 0.31 | 29.6 | 14.9 | 0.31 | 34.3 | 8.3 |
| 1 | 15.7 | 3.7 | 0.1 | 4 | 1.5 | 0.1 | 9.8 | 5.6 | 0.1 | 12.1 | 7.2 |
| 0.3163 | 6 | 5.2 | 0.0311 | 5.5 | 3.3 | 0.0311 | 11.8 | 0.7 | 0.0311 | 6.1 | 1.7 |

*concentration refers to the concentration of staurosporine in the mixture of compounds, the concentration of 17AAG results from the molar ratio used

TABLE 21b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 1.4 | 1.26 | 1.17 | 1.11 | 1.06 | 1.01 | 0.98 | 0.94 | 0.91 | 0.88 | 0.85 | 0.82 | 0.79 | 0.76 | 0.73 | 0.69 | 0.65 |
| CZAST | | | | | | | | | | | | | | | | | |
| CI | 0.9 | 0.89 | 0.85 | 0.82 | 0.8 | 0.77 | 0.76 | 0.74 | 0.72 | 0.7 | 0.69 | 0.67 | 0.66 | 0.64 | 0.62 | 0.6 | 0.57 |
| Nano | | | | | | | | | | | | | | | | | |

TABLE 22a
Inhibition of cell profiling of line 5MGBA for individual concentrations of 17AAG,
Staurosporine and their combinations at a ratio of 5/1 including standard deviation
Mobile line: 8MGBA

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 4T]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 1000 | 81.1 | 9.8 | 100 | 94.5 | 2.6 | 100 | 85.9 | 14.4 | 100 | 97.7 | 0.5 |
| 316.3 | 64.6 | 7.1 | 30 | 92.4 | 7.8 | 30 | 81.5 | 23.8 | 30 | 92.6 | 7.1 |
| 100 | 37.4 | 4.2 | 10 | 92.2 | 6.2 | 10 | 75.8 | 27.7 | 10 | 94.2 | 2.9 |
| 31.6 | 20.1 | 6 | 3.1 | 73.4 | 3.4 | 3.1 | 49.5 | 18.3 | 3.1 | 74 | 4 |
| 10 | 7.8 | 5.9 | 1 | 32.5 | 7.9 | 1 | 24.7 | 0.1 | 1 | 51 | 11.9 |
| 3.2 | 4.7 | 5.5 | 0.31 | 13.9 | 5.3 | 0.31 | 11.1 | 10.6 | 0.31 | 24.1 | 5.4 |
| 1 | 15.7 | 3.7 | 0.1 | 4 | 1.5 | 0.1 | 7 | 11 | 0.1 | 6.8 | 3.7 |
| 0.3163 | 6 | 5.2 | 0.0311 | 5.5 | 3.3 | 0.0311 | 7.3 | 5.2 | 0.0311 | 4.8 | 5.7 |

*concentration refers to the concentration of staurosporine in the mixture of compounds, the concentration of 17AAG results from the molar ratio used

TABLE 22b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 1 | 1.01 | 1.01 | 1.02 | 1.02 | 1.02 | 1.02 | 1.03 | 1.03 | 1.03 | 1.03 | 1.03 | 1.04 | 1.04 | 1.04 | 1.05 | 1.05 |
| CZAST | | | | | | | | | | | | | | | | | |
| CI | 0.9 | 0.9 | 0.92 | 0.93 | 0.94 | 0.95 | 0.97 | 0.98 | 0.99 | 1 | 1.01 | 1.03 | 1.04 | 1.06 | 1.08 | 1.1 | 1.14 |
| Nano | | | | | | | | | | | | | | | | | |

TABLE 23a
Inhibition of Hs683 line cell profiling for individual concentrations of 17AAG,
Staurosporine and their combinations at a ratio of 5/1 including standard deviation
Mobile line: Hs683

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 1G] 1/5 | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 1000 | 94.3 | 3.1 | 100 | 98.7 | 2.2 | 100 | 94.1 | 8.4 | 100 | 100.2 | 1.3 |
| 316.3 | 91 | 6.8 | 30 | 97.7 | 5.4 | 30 | 93.5 | 7.8 | 30 | 100.1 | 1.1 |
| 100 | 82 | 12.9 | 10 | 98.7 | 2.8 | 10 | 86.3 | 11.4 | 10 | 95.9 | 2.5 |
| 31.6 | 79.2 | 22.7 | 3.1 | 85.7 | 18.9 | 3.1 | 50.2 | 10.9 | 3.1 | 69.6 | 2.4 |
| 10 | 93.9 | 8.6 | 1 | 19.8 | 5.9 | 1 | 19.1 | 6 | 1 | 45.4 | 4.4 |
| 3.2 | 63 | 5 | 0.31 | 8.4 | 5.4 | 0.31 | 5.9 | 6.6 | 0.31 | 27.6 | 4.5 |
| 1 | 50.3 | 1.8 | 0.1 | −1.7 | 2.1 | 0.1 | −5.5 | 3.3 | 0.1 | 11 | 5.6 |
| 0.3163 | 8.3 | 6.3 | 0.0311 | 0.7 | 3.5 | 0.0311 | 1.4 | 6.6 | 0.0311 | 4.2 | 2.5 |

*concentration refers to the concentration of staurosporine in the mixture of compounds, the concentration of 17AAG results from the molar ratio used

TABLE 23b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 0.7 | 0.77 | 0.8 | 0.84 | 0.87 | 0.9 | 0.95 | 0.99 | 1.04 | 1.09 | 1.15 | 1.23 | 1.32 | 1.44 | 1.61 | 1.86 | 2.33 |
| CZAST | | | | | | | | | | | | | | | | | |
| CI | 0.7 | 0.67 | 0.69 | 0.7 | 0.72 | 0.73 | 0.75 | 0.77 | 0.79 | 0.82 | 0.86 | 0.9 | 0.96 | 1.04 | 1.17 | 1.39 | 1.86 |
| Nano | | | | | | | | | | | | | | | | | |

TABLE 24a
Inhibition of cell prophylation of line Hs683 for individual concentrations of 17AAG,
Staurosporine and their combinations at a ratio of 5/1 including standard deviation
Mobile line: Hs683

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 3G]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 1000 | 94.3 | 3.1 | 100 | 98.7 | 2.2 | 100 | 100.1 | 0.2 | 100 | 100.6 | 1.2 |
| 316.3 | 91 | 6.8 | 30 | 100.7 | 0.8 | 30 | 99.3 | 1.7 | 30 | 100.5 | 1.1 |
| 100 | 82 | 12.9 | 10 | 98.7 | 2.8 | 10 | 94.3 | 7.2 | 10 | 97.9 | 3.2 |
| 31.6 | 79.2 | 22.7 | 3.1 | 85.7 | 18.9 | 3.1 | 79.1 | 2.9 | 3.1 | 86.4 | 8 |
| 10 | 93.9 | 8.6 | 1 | 19.8 | 5.9 | 1 | 34.9 | 2.2 | 1 | 45 | 0.6 |
| 3.2 | 63 | 5 | 0.31 | 8.4 | 5.4 | 0.31 | 15.8 | 1.2 | 0.31 | 24.8 | 3.8 |
| 1 | 50.3 | 1.8 | 0.1 | −1.7 | 2.1 | 0.1 | 5.6 | 3.7 | 0.1 | 13.7 | 0 |
| 0.3163 | 8.3 | 6.3 | 0.0311 | 0.7 | 3.5 | 0.0311 | 3.3 | 0 | 0.0311 | 2.1 | 1.4 |

*concentration refers to the concentration of staurosporine in the mixture of compounds, the concentration of 17AAG results from the molar ratio used

TABLE 24b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 0.8 | 0.83 | 0.88 | 0.92 | 0.96 | 0.99 | 1.03 | 1.06 | 1.1 | 1.14 | 1.18 | 1.22 | 1.27 | 1.33 | 1.4 | 1.49 | 1.62 |
| CZAST | | | | | | | | | | | | | | | | | |
| CI | 0.7 | 0.66 | 0.64 | 0.62 | 0.61 | 0.6 | 0.6 | 0.6 | 0.6 | 0.6 | 0.61 | 0.62 | 0.64 | 0.66 | 0.71 | 0.78 | 0.93 |
| Nano | | | | | | | | | | | | | | | | | |

TABLE 25a
Inhibition of Hs683 line cell profiling for individual concentrations of 17AAG,
Staurosporine and their combinations at a ratio of 5/1 including standard deviation
Mobile line: Hs683

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 4T]. | | |
| Concentration | | | Concentration | | | Concentration* | | | Concentration* | | |
| [nM] | Zah. | SD | [nM] | Zah. | SD | [nM] | Zah. | SD | [nM] | Zah | SD |
| 1000 | 94.3 | 3.1 | 100 | 98.7 | 2.2 | 100 | 95.7 | 5.6 | 100 | 100.5 | 1.6 |
| 316.3 | 91 | 6.8 | 30 | 94.3 | 11.1 | 30 | 92.4 | 10.1 | 30 | 99.4 | 1.3 |
| 100 | 82 | 12.9 | 10 | 98.7 | 2.8 | 10 | 92.7 | 4.8 | 10 | 93.8 | 4.6 |
| 31.6 | 79.2 | 22.7 | 3.1 | 85.7 | 18.9 | 3.1 | 62.8 | 1.7 | 3.1 | 64.8 | 9.9 |
| 10 | 93.9 | 8.6 | 1 | 19.8 | 5.9 | 1 | 23.6 | 5.2 | 1 | 34.7 | 4.4 |
| 3.2 | 63 | 5 | 0.31 | 8.4 | 5.4 | 0.31 | −1.5 | 6.5 | 0.31 | 11.9 | 2.9 |
| 1 | 50.3 | 1.8 | 0.1 | −1.7 | 2.1 | 0.1 | −3 | 2.4 | 0.1 | −1.9 | 2.8 |
| 0.3163 | 8.3 | 6.3 | 0.0311 | 0.7 | 3.5 | 0.0311 | 0.1 | 11.4 | 0.0311 | −1.5 | 1.4 |

*concentration refers to the concentration of staurosporine in the mixture of compounds, the concentration of 17AAG results from the molar ratio used

TABLE 25b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 0.6 | 0.83 | 0.98 | 1.11 | 1.23 | 1.34 | 1.45 | 1.55 | 1.66 | 1.76 | 1.87 | 1.99 | 2.13 | 2.28 | 2.45 | 2.68 | 2.99 |
| CZAST | | | | | | | | | | | | | | | | | |
| CI | 0.8 | 1.03 | 1.18 | 1.31 | 1.43 | 1.54 | 1.65 | 1.75 | 1.86 | 1.96 | 2.07 | 2.19 | 2.33 | 2.48 | 2.65 | 2.88 | 3.19 |
| Nano | | | | | | | | | | | | | | | | | |

TABLE 26a
Inhibition of LN229 line cell profiling for individual concentrations of 17AAG,
Staurosporine and their combinations at a ratio of 5/1 including standard deviation
Mobile line: LN229

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 1G]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 10000.5 | 81.5 | 1.2 | 1000 | 91.3 | 3.9 | 1000 | 91.2 | 3.7 | 1000 | 102.6 | 6.5 |
| 3162.5 | 71.6 | 6.8 | 316.3 | 48.3 | 0.5 | 316.3 | 71.4 | 0.6 | 316.3 | 98.9 | 5.3 |
| 1000 | 62.9 | 11.2 | 100 | 56.2 | 4.7 | 100 | 53.4 | 3.2 | 100 | 74.8 | 1.7 |
| 316.3 | 38.7 | 11.1 | 31.6 | 44.7 | 2.5 | 31.6 | 47.5 | 3.3 | 31.6 | 51.1 | 2.2 |
| 100 | 28.9 | 10.7 | 10 | 39.4 | 2.9 | 10 | 42.7 | 6.9 | 10 | 65.2 | 10.2 |
| 31.6 | 18.4 | 18.9 | 3.2 | 38.3 | 4.2 | 3.2 | 34.9 | 3.6 | 3.2 | 45 | 1.3 |
| 10 | 3.1 | 4.2 | 1 | 31.9 | 0.9 | 1 | 30.4 | 2.6 | 1 | 35.1 | 1.5 |
| 3.2 | 3.3 | 4.1 | 0.3163 | 28.3 | 3.8 | 0.3163 | 23.9 | 3.9 | 0.3163 | 30.7 | 3.4 |

*concentration refers to the concentration of staurosporine in the mixture of compounds, the concentration of 17AAG results from the molar ratio used

TABLE 26b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 1 | 0.98 | 0.96 | 0.95 | 0.93 | 0.92 | 0.91 | 0.89 | 0.88 | 0.87 | 0.86 | 0.85 | 0.84 | 0.82 | 0.81 | 0.79 | 0.77 |
| CZAST | | | | | | | | | | | | | | | | | |
| CI | 0.7 | 0.68 | 0.69 | 0.7 | 0.71 | 0.71 | 0.72 | 0.73 | 0.74 | 0.74 | 0.75 | 0.76 | 0.77 | 0.78 | 0.8 | 0.82 | 0.84 |
| Nano | | | | | | | | | | | | | | | | | |

TABLE 27a
Inhibition of LN229 line cell profiling for individual concentrations of 17AAG,
Staurosporine and their combinations at a ratio of 5/1 including standard deviation
Mobile line: LN229

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 3G]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 10000.5 | 81.5 | 1.2 | 1000 | 91.3 | 3.9 | 1000 | 93.4 | 4.1 | 1000 | 93 | 4.2 |
| 3162.5 | 71.6 | 6.8 | 316.3 | 55.8 | 10 | 316.3 | 81.4 | 5.7 | 316.3 | 88.6 | 4.8 |
| 1000 | 62.9 | 11.2 | 100 | 56.2 | 4.7 | 100 | 70.2 | 5 | 100 | 69.5 | 4.6 |
| 316.3 | 38.7 | 11.1 | 31.6 | 47.1 | 4.5 | 31.6 | 60.4 | 4 | 31.6 | 55.2 | 9.1 |
| 100 | 28.9 | 10.7 | 10 | 39.4 | 2.9 | 10 | 49.8 | 9.7 | 10 | 53.8 | 5.2 |
| 31.6 | 18.4 | 18.9 | 3.2 | 38.3 | 4.2 | 3.2 | 35.8 | 4.6 | 3.2 | 40.8 | 7.2 |
| 10 | 3.1 | 4.2 | 1 | 31.9 | 0.9 | 1 | 33.7 | 2.3 | 1 | 31.5 | 0.5 |
| 3.2 | 3.3 | 4.1 | 0.3163 | 28.3 | 3.8 | 0.3163 | 31.1 | 0 | 0.3163 | 24.5 | 1.2 |

*concentration refers to the concentration of staurosporine in the mixture of compounds, the concentration of 17AAG results from the molar ratio used

TABLE 27b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 1 | 0.89 | 0.83 | 0.78 | 0.75 | 0.72 | 0.7 | 0.69 | 0.68 | 0.67 | 0.67 | 0.67 | 0.67 | 0.69 | 0.71 | 0.75 | 0.84 |
| CZAST | | | | | | | | | | | | | | | | | |
| CI | 1.3 | 1.11 | 1.01 | 0.94 | 0.9 | 0.86 | 0.83 | 0.81 | 0.8 | 0.78 | 0.78 | 0.78 | 0.78 | 0.79 | 0.81 | 0.84 | 0.9 |
| Nano | | | | | | | | | | | | | | | | | |

TABLE 28a
Inhibition of LN229 line cell profiling for individual concentrations of 17AAG,
Staurosporine and their combinations at a ratio of 5/1 including standard deviation
Mobile line: LN229

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 4T]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 10000.5 | 81.5 | 1.2 | 1000 | 91.3 | 3.9 | 1000 | 94.7 | 3 | 1000 | 101.4 | 1.2 |
| 3162.5 | 71.6 | 6.8 | 316.3 | 48.3 | 0.5 | 316.3 | 64.6 | 9.6 | 316.3 | 88.4 | 8 |
| 1000 | 62.9 | 11.2 | 100 | 56.2 | 4.7 | 100 | 49.8 | 5.4 | 100 | 65.2 | 3.8 |
| 316.3 | 38.7 | 11.1 | 31.6 | 47.1 | 4.5 | 31.6 | 50 | 6 | 31.6 | 48.4 | 2.3 |
| 100 | 28.9 | 10.7 | 10 | 39.4 | 2.9 | 10 | 41.6 | 5.5 | 10 | 39.1 | 0 |
| 31.6 | 18.4 | 18.9 | 3.2 | 38.3 | 4.2 | 3.2 | 29.5 | 6.6 | 3.2 | 32.5 | 4.6 |
| 10 | 3.1 | 4.2 | 1 | 31.9 | 0.9 | 1 | 27.9 | 4.8 | 1 | 20.4 | 1.7 |
| 3.2 | 3.3 | 4.1 | 0.3163 | 28.3 | 3.8 | 0.3163 | 24.2 | 6.1 | 0.3163 | 14.5 | 4.3 |

*concentration refers to the concentration of staurosporine in the mixture of compounds, the concentration of 17AAG results from the molar ratio used

TABLE 28b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 0.9 | 0.92 | 0.94 | 0.95 | 0.96 | 0.96 | 0.97 | 0.98 | 0.99 | 0.99 | 1 | 1.01 | 1.02 | 1.03 | 1.04 | 1.05 | 1.07 |
| CZAST | | | | | | | | | | | | | | | | | |
| CI | 1.2 | 1.12 | 1.09 | 1.07 | 1.05 | 1.04 | 1.03 | 1.01 | 1 | 0.99 | 0.97 | 0.96 | 0.95 | 0.93 | 0.91 | 0.89 | 0.87 |
| Nano | | | | | | | | | | | | | | | | | |

TABLE 29a
Inhibition of LUDLU-1 line cell profiling for individual concentrations of 17AAG,
Staurosporine and their combinations at a ratio of 5/1 including standard deviation
Cell line: LUDLU-1

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 1G]. | | |
| Concentration | | | Concentration | | | Concentration* | | | Concentration* | | |
| [M] | Zah. | SD | [nM] | Zah. | SD | [nM] | Zah. | SD | [nM] | Zah. | SD |
| 1000 | 80.2 | 0.8 | 100 | 101.4 | 3.8 | 100 | 93.1 | 15.2 | 100 | 100.9 | 3.4 |
| 316.3 | 76.9 | 1.9 | 31.6 | 98.8 | 2.8 | 31.6 | 81 | 19.9 | 31.6 | 96.4 | 9.5 |
| 100 | 65.7 | 6.1 | 10 | 79.1 | 5.6 | 10 | 64.5 | 10.3 | 10 | 78.9 | 0.4 |
| 31.6 | 60.2 | 17.4 | 3.2 | 51.7 | 9.1 | 3.2 | 51.4 | 8.4 | 3.2 | 53.6 | 6.5 |
| 10 | 75.1 | 0.5 | 1 | 40.6 | 8.7 | 1 | 41.9 | 8.4 | 1 | 55.3 | 3.5 |
| 3.2 | 61.9 | 0.1 | 0.3163 | 28.1 | 6.1 | 0.3163 | 28.4 | 2.5 | 0.3163 | 39.4 | 2.4 |
| 1 | 29.3 | 4.9 | 0.1 | 12.5 | 8.9 | 0.1 | 27.8 | 2.7 | 0.1 | 23.6 | 3.7 |
| 0.3163 | 3.9 | 6.4 | 0.0316 | 12.1 | 4 | 0.0316 | 13.1 | 7.2 | 0.0316 | 8.3 | 3.3 |

*concentration refers to the concentration of staurosporine in the mixture of compounds, the concentration of 17AAG results from the molar ratio used

TABLE 29b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 0.7 | 0.75 | 0.78 | 0.81 | 0.84 | 0.86 | 0.89 | 0.91 | 0.93 | 0.96 | 0.99 | 1.01 | 1.05 | 1.08 | 1.12 | 1.18 | 1.26 |
| CZAST | | | | | | | | | | | | | | | | | |
| CI | 0.9 | 0.87 | 0.84 | 0.83 | 0.81 | 0.8 | 0.79 | 0.78 | 0.77 | 0.76 | 0.75 | 0.74 | 0.73 | 0.72 | 0.71 | 0.7 | 0.68 |
| Nano | | | | | | | | | | | | | | | | | |

TABLE 30a
Inhibition of LUDLU-1 line cell profiling for individual concentrations of 17AAG,
Staurosporine and their combinations at a ratio of 5/1 including standard deviation
Cell line: LUDLU-1

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 3G]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 1000 | 80.2 | 0.8 | 100 | 101.4 | 3.8 | 100 | 100.2 | 3.7 | 100 | 100.7 | 3.3 |
| 316.3 | 76.9 | 1.9 | 31.6 | 98.8 | 2.8 | 31.6 | 100.2 | 2.8 | 31.6 | 98.9 | 5.6 |
| 100 | 65.7 | 6.1 | 10 | 79.1 | 5.6 | 10 | 75 | 0.6 | 10 | 69.8 | 11.8 |
| 31.6 | 60.2 | 17.4 | 3.2 | 51.7 | 9.1 | 3.2 | 56.2 | 4.8 | 3.2 | 58.2 | 8.2 |
| 10 | 75.1 | 0.5 | 1 | 40.6 | 8.7 | 1 | 45.8 | 4.7 | 1 | 53.6 | 5.5 |
| 3.2 | 61.9 | 0.1 | 0.3163 | 28.1 | 6.1 | 0.3163 | 27.7 | 0.4 | 0.3163 | 39 | 1.2 |
| 1 | 29.3 | 4.9 | 0.1 | 22.3 | 5 | 0.1 | 21 | 7.1 | 0.1 | 19.6 | 0.7 |
| 0.3163 | 3.9 | 6.4 | 0.0316 | 9.9 | 1.2 | 0.0316 | 10.7 | 6.3 | 0.0316 | 9.8 | 3.4 |

*concentration refers to the concentration of staurosporine in the mixture of compounds, the concentration of 17AAG results from the molar ratio used

TABLE 30b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 1.1 | 0.97 | 0.89 | 0.84 | 0.8 | 0.79 | 0.78 | 0.77 | 0.76 | 0.76 | 0.76 | 0.76 | 0.76 | 0.77 | 0.78 | 0.79 | 0.81 |
| CZAST | | | | | | | | | | | | | | | | | |
| CI | 0.9 | 0.87 | 0.85 | 0.85 | 0.84 | 0.83 | 0.82 | 0.82 | 0.81 | 0.81 | 0.8 | 0.8 | 0.79 | 0.78 | 0.77 | 0.77 | 0.75 |
| Nano | | | | | | | | | | | | | | | | | |

TABLE 31a
Inhibition of LUDLU-1 line cell profiling for individual concentrations of 17AAG,
Staurosporine and their combinations at a ratio of 5/1 including standard deviation
Cell line: LUDLU-1

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 4T]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 1000 | 80.2 | 0.8 | 100 | 101.4 | 3.8 | 100 | 90.2 | 18.5 | 100 | 99.7 | 2.8 |
| 316.3 | 76.9 | 1.9 | 31.6 | 98.8 | 2.8 | 31.6 | 81.8 | 16.8 | 31.6 | 100.2 | 1.3 |
| 100 | 65.7 | 6.1 | 10 | 79.1 | 5.6 | 10 | 62.3 | 13.6 | 10 | 74.6 | 0.4 |
| 31.6 | 60.2 | 17.4 | 3.2 | 51.7 | 9.1 | 3.2 | 49.8 | 11 | 3.2 | 58.4 | 3.9 |
| 10 | 75.1 | 0.5 | 1 | 40.6 | 8.7 | 1 | 41.8 | 6.1 | 1 | 53.2 | 0.6 |
| 3.2 | 61.9 | 0.1 | 0.3163 | 25.1 | 4.5 | 0.3163 | 23 | 9.3 | 0.3163 | 29.5 | 5.3 |
| 1 | 29.3 | 4.9 | 0.1 | 12.5 | 8.9 | 0.1 | 16.3 | 1.5 | 0.1 | 7.8 | 7.8 |
| 0.3163 | 3.9 | 6.4 | 0.0316 | 9.9 | 1.2 | 0.0316 | 8.8 | 6 | 0.0316 | 5.2 | 2.1 |

*concentration refers to the concentration of staurosporine in the mixture of compounds, the concentration of 17AAG results from the molar ratio used

TABLE 31b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 1.2 | 1.13 | 1.06 | 1.01 | 0.97 | 0.94 | 0.92 | 0.9 | 0.89 | 0.88 | 0.87 | 0.86 | 0.86 | 0.87 | 0.87 | 0.9 | 0.94 |
| CZAST | | | | | | | | | | | | | | | | | |
| CI | 1.4 | 1.31 | 1.25 | 1.2 | 1.16 | 1.13 | 1.09 | 1.07 | 1.04 | 1.01 | 0.99 | 0.96 | 0.94 | 0.92 | 0.9 | 0.9 | 0.92 |
| Nano | | | | | | | | | | | | | | | | | |

TABLE 32a
Inhibition of HCI-H226 line cell profiling for individual concentrations of 17AAG,
Staurosporine and their combinations at a ratio of 5/1 including standard deviation
Cell line: NCI-H226

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 1G]. | | |
| Concentration | | | Concentration | | | Concentration* | | | Concentration* | | |
| [nM] | Zah | SD | [nM] | Zah. | SD | [nM] | Zah. | SD | [nM] | Zah. | SD |
| 10000.5 | 61.4 | 11.3 | 1000 | 100.4 | 1 | 1000 | 94.6 | 4.7 | 1000 | 100.5 | 0.6 |
| 3162.5 | 63.5 | 5.9 | 316.3 | 88.5 | 4.7 | 316.3 | 66.4 | 2.8 | 316.3 | 100.2 | 0.5 |
| 1000 | 59 | 4.3 | 100 | 83.9 | 7.1 | 100 | 54.2 | 4.8 | 100 | 95.5 | 4.2 |
| 316.3 | 49.3 | 9.8 | 31.6 | 41.1 | 6.7 | 31.6 | 42.8 | 9.3 | 31.6 | 83.6 | 3.6 |
| 100 | 44.7 | 5.1 | 10 | 26.7 | 5.1 | 10 | 35.1 | 10.1 | 10 | 66.9 | 1 |
| 31.6 | 30.6 | 3.7 | 3.2 | 15.1 | 0.3 | 3.2 | 32.3 | 1.4 | 3.2 | 39.1 | 2.2 |
| 10 | −4.6 | 0.3 | 1 | 1.2 | 3.7 | 1 | 3.7 | 1.2 | 1 | 14 | 1.3 |
| 3.2 | −6.6 | 0 | 0.3163 | 8.4 | 3.4 | 0.3163 | 1.9 | 3.2 | 0.3163 | 5.4 | 1.7 |

*concentration refers to the concentration of staurosporine in the mixture of compounds, the concentration of 17AAG results from the molar ratio used

TABLE 32b
Values of affected fractions and corresponding combination index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 0.7 | 0.7 | 0.73 | 0.76 | 0.79 | 0.81 | 0.84 | 0.86 | 0.88 | 0.91 | 0.94 | 0.96 | 1 | 1.03 | 1.07 | 1.13 | 1.21 |
| CZAST | | | | | | | | | | | | | | | | | |
| CI | 1.1 | 1.1 | 1.01 | 0.79 | 0.63 | 0.52 | 0.44 | 0.37 | 0.32 | 0.28 | 0.24 | 0.21 | 0.18 | 0.16 | 0.14 | 0.12 | 0.11 |
| Nano | | | | | | | | | | | | | | | | | |

TABLE 33a
Inhibition of cell prophylation of HCI-H226 lines for individual concentrations of 17AAG,
Staurosporine and their combinations at a ratio of 5/1 including standard deviation
Cell line: NCI-H226

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 3G]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 10000.5 | 61.4 | 11.3 | 1000 | 100.4 | 1 | 1000 | 100.6 | 0.8 | 1000 | 100.5 | 0.9 |
| 3162.5 | 63.5 | 5.9 | 316.3 | 88.5 | 4.7 | 316.3 | 99.4 | 0.7 | 316.3 | 97.5 | 4.3 |
| 1000 | 59 | 4.3 | 100 | 83.9 | 7.1 | 100 | 96.2 | 2.7 | 100 | 95.9 | 3.1 |
| 316.3 | 49.3 | 9.8 | 31.6 | 41.1 | 6.7 | 31.6 | 63 | 2.4 | 31.6 | 71.8 | 17.1 |
| 100 | 44.7 | 5.1 | 10 | 26.7 | 5.1 | 10 | 47.3 | 4.3 | 10 | 49.2 | 1.6 |
| 31.6 | 30.6 | 3.7 | 3.2 | 11.1 | 6.9 | 3.2 | 38.5 | 5.8 | 3.2 | 40.4 | 1.8 |
| 10 | −4.6 | 0.3 | 1 | 1.2 | 3.7 | 1 | 23.2 | 2.6 | 1 | 21.6 | 3.1 |
| 3.2 | −6.6 | 0 | 0.3163 | 8.4 | 3.4 | 0.3163 | 9.6 | 6 | 0.3163 | 1.9 | 1.5 |

*concentration refers to the concentration of staurosporine in the mixture of compounds, the concentration of 17AAG results from the molar ratio used

TABLE 33b
Values of affected fractions and corresponding combination
index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 1.1 | 1.38 | 1.01 | 0.79 | 0.63 | 0.52 | 0.44 | 0.37 | 0.32 | 0.28 | 0.24 | 0.21 | 0.18 | 0.16 | 0.14 | 0.12 | 0.11 |
| CZAST | | | | | | | | | | | | | | | | | |
| CI | 1.4 | 1.06 | 0.87 | 0.74 | 0.65 | 0.57 | 0.51 | 0.46 | 0.41 | 0.37 | 0.34 | 0.31 | 0.28 | 0.25 | 0.23 | 0.22 | 0.22 |
| Nano | | | | | | | | | | | | | | | | | |

TABLE 34a
Inhibition of cell prophylation of HCI-H226 lines for individual
concentrations of 17AAG, Staurosporine and their combinations at a ratio
of 5/1 including standard deviation
Cell line: NCI-H226

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 4T]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 10000.5 | 61.4 | 11.3 | 1000 | 100.4 | 1 | 1000 | 97.5 | 2.4 | 1000 | 100.5 | 1.3 |
| 3162.5 | 63.5 | 5.9 | 316.3 | 88.5 | 4.7 | 316.3 | 67.6 | 1.3 | 316.3 | 93.4 | 9.1 |
| 1000 | 59 | 4.3 | 100 | 83.9 | 7.1 | 100 | 60 | 4.2 | 100 | 95.8 | 1.3 |
| 316.3 | 49.3 | 9.8 | 31.6 | 41.1 | 6.7 | 31.6 | 41 | 1.4 | 31.6 | 41.4 | 4.4 |
| 100 | 44.7 | 5.1 | 10 | 26.7 | 5.1 | 10 | 36.3 | 2.2 | 10 | 45.8 | 3.4 |
| 31.6 | 30.6 | 3.7 | 3.2 | 11.1 | 6.9 | 3.2 | 29.8 | 2.9 | 3.2 | 14.7 | 5.4 |
| 10 | −4.6 | 0.3 | 1 | 5.2 | 7.4 | 1 | 9.8 | 6.9 | 1 | −1.5 | 5.1 |
| 3.2 | −6.6 | 0 | 0.3163 | 8.4 | 3.4 | 0.3163 | 0.8 | 6.3 | 0.3163 | 4.5 | 1.5 |

*- concentration refers to the concentration of staurosporine in the mixture of compounds, the concentration of 17AAG
results from the molar ratio used

TABLE 34b
Values of affected fractions and corresponding combination
index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 1.1 | 1 | 0.98 | 0.99 | 1.03 | 1.08 | 1.14 | 1.23 | 1.32 | 1.44 | 1.59 | 1.78 | 2.02 | 2.33 | 2.78 | 3.47 | 4.7 |
| CZAST | | | | | | | | | | | | | | | | | |
| CI | 0.6 | 0.6 | 0.63 | 0.66 | 0.69 | 0.71 | 0.74 | 0.76 | 0.78 | 0.81 | 0.84 | 0.86 | 0.9 | 0.93 | 0.97 | 1.03 | 1.11 |
| Nano | | | | | | | | | | | | | | | | | |

TABLE 35a
Inhibition of cell prophylation of HCI-H226 lines for individual
concentrations of 17AAG, Staurosporine and their combinations at a ratio
of 5/1 including standard deviation
Cell line: NCI-H226

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 1G]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 10000.5 | 68.4 | 3.1 | 1000 | 100.7 | 1 | 1000 | 91.9 | 7.2 | 1000 | 100.7 | 1.1 |
| 3162.5 | 69.5 | 3.5 | 316.3 | 92.1 | 3.2 | 316.3 | 86.7 | 11.3 | 316.3 | 100.8 | 1.4 |
| 1000 | 65 | 1.6 | 100 | 77.1 | 8.7 | 100 | 94.6 | 4.9 | 100 | 99.3 | 1.6 |
| 316.3 | 59.1 | 5.1 | 31.6 | 46.8 | 7.9 | 31.6 | 53.1 | 18.1 | 31.6 | 87.6 | 10.1 |
| 100 | 51.4 | 4.3 | 10 | 23.4 | 5.1 | 10 | 45.4 | 2.2 | 10 | 56.8 | 8.4 |
| 31.6 | 29.4 | 9.6 | 3.2 | 10.8 | 1.7 | 3.2 | 3.9 | 4.1 | 3.2 | 29.3 | 5.6 |
| 10 | 17.2 | 15.5 | 1 | 2.4 | 1 | 1 | 3.9 | 4.5 | 1 | 9.2 | 3.2 |
| 3.2 | 10.4 | 4.4 | 0.3163 | −3.6 | 1.8 | 0.3163 | −3.8 | 6.4 | 0.3163 | −6.9 | 1.9 |

*concentration refers to the concentration of staurosporine in the mixture of compounds, the concentration of 17AAG results from the molar ratio used

TABLE 35b
Values of affected fractions and corresponding combination
index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 0.7 | 0.7 | 0.74 | 0.78 | 0.81 | 0.84 | 0.87 | 0.89 | 0.92 | 0.95 | 0.98 | 1.02 | 1.06 | 1.1 | 1.15 | 1.22 | 1.31 |
| CZAST | | | | | | | | | | | | | | | | | |
| CI | 0.7 | 0.72 | 0.71 | 0.7 | 0.69 | 0.69 | 0.68 | 0.68 | 0.67 | 0.67 | 0.67 | 0.66 | 0.66 | 0.65 | 0.65 | 0.65 | 0.64 |
| Nano | | | | | | | | | | | | | | | | | |

TABLE 36a
Inhibition of cell prophylation of HCI-H226 lines for individual
concentrations of 17AAG, Staurosporine and their combinations at a ratio
of 5/1 including standard deviation
Cell line: NCI-H226

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 3G]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 10000.5 | 68.4 | 3.1 | 1000 | 100.7 | 1 | 1000 | 100.8 | 0.9 | 1000 | 100.7 | 1.1 |
| 3162.5 | 69.5 | 3.5 | 316.3 | 92.1 | 3.2 | 316.3 | 100 | 1.9 | 316.3 | 100.9 | 0.8 |
| 1000 | 65 | 1.6 | 100 | 77.1 | 8.7 | 100 | 98.4 | 3.6 | 100 | 98.7 | 3.8 |
| 316.3 | 59.1 | 5.1 | 31.6 | 46.8 | 7.9 | 31.6 | 85.6 | 13.2 | 31.6 | 92.2 | 7.1 |
| 100 | 51.4 | 4.3 | 10 | 23.4 | 5.1 | 10 | 54.8 | 4.5 | 10 | 73.4 | 3.5 |
| 31.6 | 29.4 | 9.6 | 3.2 | 10.8 | 1.7 | 3.2 | 35 | 5.5 | 3.2 | 40.1 | 1.6 |
| 10 | 17.2 | 15.5 | 1 | 2.4 | 1 | 1 | 8.9 | 2.1 | 1 | 6.7 | 4 |
| 3.2 | 10.4 | 4.4 | 0.3163 | −3.6 | 1.8 | 0.3163 | 4.8 | 4.3 | 0.3163 | −2.6 | 1.4 |

*concentration refers to the concentration of staurosporine in the mixture of compounds, the concentration of 17AAG results from the molar ratio used

TABLE 36b
Values of affected fractions and corresponding combination
index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 0.2 | 0.23 | 0.27 | 0.31 | 0.34 | 0.38 | 0.41 | 0.45 | 0.49 | 0.53 | 0.58 | 0.63 | 0.7 | 0.78 | 0.88 | 1.01 | 1.23 |
| CZAST | | | | | | | | | | | | | | | | | |
| CI | 0.3 | 0.31 | 0.33 | 0.35 | 0.36 | 0.37 | 0.39 | 0.4 | 0.41 | 0.43 | 0.44 | 0.46 | 0.48 | 0.5 | 0.53 | 0.56 | 0.61 |
| Nano | | | | | | | | | | | | | | | | | |

TABLE 37a
Inhibition of cell prophylation of HCI-H226 lines for individual
concentrations of 17AAG, Staurosporine and their combinations at a ratio
of 5/1 including standard deviation
Cell line: NCI-H226

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 4T]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 10000.5 | 68.4 | 3.1 | 1000 | 100.7 | 1 | 1000 | 93.5 | 6.1 | 1000 | 100.3 | 1.2 |
| 3162.5 | 69.5 | 3.5 | 316.3 | 92.1 | 3.2 | 316.3 | 84.6 | 16.3 | 316.3 | 100 | 1.3 |
| 1000 | 65 | 1.6 | 100 | 77.1 | 8.7 | 100 | 63 | 5.8 | 100 | 95 | 5 |
| 316.3 | 59.1 | 5.1 | 31.6 | 46.8 | 7.9 | 31.6 | 53.9 | 5.9 | 31.6 | 75.7 | 14.8 |
| 100 | 51.4 | 4.3 | 10 | 23.4 | 5.1 | 10 | 39.8 | 6.4 | 10 | 40.7 | 10.7 |
| 31.6 | 29.4 | 9.6 | 3.2 | 10.8 | 1.7 | 3.2 | 14 | 10.5 | 3.2 | 5.8 | 1.9 |
| 10 | 17.2 | 15.5 | 1 | 2.4 | 1 | 1 | −2.8 | 9.6 | 1 | −4.7 | 5.7 |
| 3.2 | 10.4 | 4.4 | 0.3163 | −3.6 | 1.8 | 0.3163 | −1.8 | 2.4 | 0.3163 | −4.9 | 4 |

*concentration refers to the concentration of staurosporine in the mixture of compounds, the concentration of 17AAG results from the molar ratio used

TABLE 37b
Values of affected fractions and corresponding combination
index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 1 | 0.95 | 0.92 | 0.91 | 0.89 | 0.88 | 0.87 | 0.86 | 0.86 | 0.85 | 0.84 | 0.84 | 0.83 | 0.83 | 0.82 | 0.82 | 0.82 |
| CZAST | | | | | | | | | | | | | | | | | |
| CI | 0.8 | 0.74 | 0.7 | 0.68 | 0.66 | 0.64 | 0.63 | 0.61 | 0.6 | 0.58 | 0.57 | 0.56 | 0.55 | 0.53 | 0.52 | 0.5 | 0.48 |
| Nano | | | | | | | | | | | | | | | | | |

TABLE 38a
Inhibition of cell profiling of SKMES-1 line for individual
concentrations of 17AAG, Staurosporine and their combinations at a ratio
of 5/1 including standard deviation
Cell line: SKMES-1

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 1G]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 1000 | 89.1 | 1.8 | 100 | 100.1 | 0.3 | 100 | 96.5 | 3.4 | 100 | 100.3 | 0.7 |
| 316.3 | 89.3 | 0.8 | 31.6 | 99.4 | 1.1 | 31.6 | 91.8 | 8.1 | 31.6 | 100.2 | 0.5 |
| 100 | 87.9 | 2.2 | 10 | 97.6 | 2.1 | 10 | 89.7 | 7.3 | 10 | 98.9 | 0.5 |
| 31.6 | 85.1 | 7.4 | 3.2 | 93 | 5.7 | 3.2 | 84.5 | 6.7 | 3.2 | 95.5 | 2.6 |
| 10 | 90.6 | 3.8 | 1 | 64.7 | 9.7 | 1 | 54.8 | 12.5 | 1 | 89.1 | 2.9 |
| 3.2 | 86.7 | 2.2 | 0.3163 | 38.4 | 5.3 | 0.3163 | 36.2 | 0.8 | 0.3163 | 71.2 | 4 |
| 1 | 46.7 | 5.2 | 0.1 | 19.7 | 2.9 | 0.1 | 7.9 | 12.9 | 0.1 | 38.8 | 7.2 |
| 0.3163 | 2.2 | 8.4 | 0.0316 | 7.6 | 6.4 | 0.0316 | 7.4 | 0.2 | 0.0316 | 18.6 | 6.5 |

*concentration refers to the concentration of staurosporine in the mixture of compounds, the concentration of 17AAG results from the molar ratio used

TABLE 38b
Values of affected fractions and corresponding combination
index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 1.5 | 1.4 | 1.35 | 1.32 | 1.29 | 1.27 | 1.25 | 1.23 | 1.21 | 1.19 | 1.17 | 1.15 | 1.13 | 1.11 | 1.08 | 1.05 | 1.02 |
| CZAST | | | | | | | | | | | | | | | | | |
| CI | 0.9 | 0.82 | 0.78 | 0.76 | 0.74 | 0.72 | 0.71 | 0.69 | 0.68 | 0.66 | 0.65 | 0.64 | 0.63 | 0.61 | 0.6 | 0.58 | 0.56 |
| Nano | | | | | | | | | | | | | | | | | |

TABLE 39a
Inhibition of cell profiling of SKMES-1 line for individual
concentrations of 17AAG, Staurosporine and their combinations at a ratio
of 5/1 including standard deviation
Cell line: SKMES-1

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 3G]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 1000 | 89.1 | 1.8 | 100 | 100.1 | 0.3 | 100 | 100.5 | 0.2 | 100 | 100.4 | 0.1 |
| 316.3 | 89.3 | 0.8 | 31.6 | 99.4 | 1.1 | 31.6 | 99.9 | 0.4 | 31.6 | 100.6 | 0.1 |
| 100 | 87.9 | 2.2 | 10 | 97.6 | 2.1 | 10 | 96.3 | 3 | 10 | 99.7 | 0.3 |
| 31.6 | 85.1 | 7.4 | 3.2 | 93 | 5.7 | 3.2 | 93.1 | 1.5 | 3.2 | 97.4 | 0.8 |
| 10 | 90.6 | 3.8 | 1 | 64.7 | 9.7 | 1 | 70.1 | 6.6 | 1 | 89.2 | 5.9 |
| 3.2 | 86.7 | 2.2 | 0.3163 | 38.4 | 5.3 | 0.3163 | 42.7 | 3.5 | 0.3163 | 62.2 | 7.9 |
| 1 | 46.7 | 5.2 | 0.1 | 19.7 | 2.9 | 0.1 | 33.3 | 6.6 | 0.1 | 32.8 | 4.6 |
| 0.3163 | 2.2 | 8.4 | 0.0316 | 7.6 | 6.4 | 0.0316 | 26.5 | 3.1 | 0.0316 | 7.7 | 5.8 |

*concentration refers to the concentration of staurosporine in the mixture of compounds, the concentration of 17AAG results from the molar ratio used

TABLE 39b
Values of affected fractions and corresponding combination
index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 1.1 | 1.05 | 1.02 | 1.01 | 0.99 | 0.98 | 0.97 | 0.96 | 0.96 | 0.95 | 0.94 | 0.94 | 0.93 | 0.93 | 0.92 | 0.92 | 0.92 |
| CZAST | | | | | | | | | | | | | | | | | |
| CI | 1 | 0.91 | 0.87 | 0.83 | 0.81 | 0.78 | 0.76 | 0.74 | 0.72 | 0.7 | 0.68 | 0.67 | 0.65 | 0.63 | 0.6 | 0.58 | 0.54 |
| Nano | | | | | | | | | | | | | | | | | |

TABLE 40a
Inhibition of cell profiling of SKMES-1 line for individual
concentrations of 17AAG, Staurosporine and their combinations at a ratio
of 5/1 including standard deviation
Cell line: SKMES-1

| | | | | | | Staurosporine + 17AAG | | | Staurosporine + 17AAG | | |
|---|---|---|---|---|---|---|---|---|---|---|---|
| 17AAG | | | Staurosporine | | | [Molecular]. | | | [NANO 4T]. | | |
| Concentration | | | Concentration | | | Concentration* | | | Concentration* | | |
| [nM] | Zah. | SD | [nM] | Zah. | SD | [nM] | Zah. | SD | [nM] | Zah. | SD |
| 1000 | 89.1 | 1.8 | 100 | 100.1 | 0.3 | 100 | 96 | 3.8 | 100 | 100.2 | 0.7 |
| 316.3 | 89.3 | 0.8 | 31.6 | 99.4 | 1.1 | 31.6 | 91.3 | 7.5 | 31.6 | 99.5 | 0.4 |
| 100 | 87.9 | 2.2 | 10 | 97.6 | 2.1 | 10 | 89.5 | 7.5 | 10 | 97.7 | 0.8 |
| 31.6 | 85.1 | 7.4 | 3.2 | 93 | 5.7 | 3.2 | 81.1 | 10 | 3.2 | 92.2 | 4.1 |
| 10 | 90.6 | 3.8 | 1 | 64.7 | 9.7 | 1 | 51.7 | 11.9 | 1 | 81.2 | 5.5 |
| 3.2 | 86.7 | 2.2 | 0.3163 | 38.4 | 5.3 | 0.3163 | 23.1 | 3.4 | 0.3163 | 52.6 | 9.9 |
| 1 | 46.7 | 5.2 | 0.1 | 19.7 | 2.9 | 0.1 | 6.9 | 8 | 0.1 | 26.1 | 1.4 |
| 0.3163 | 2.2 | 8.4 | 0.0316 | 7.6 | 6.4 | 0.0316 | 0.5 | 9.2 | 0.0316 | 4.8 | 0.7 |

*concentration refers to the concentration of staurosporine in the mixture of compounds, the concentration of 17AAG results from the molar ratio used

TABLE 40b
Values of affected fractions and corresponding combination
index obtained using CalcuSyn software

| | | | | | | | | | | | | | | | | | |
|---|---|---|---|---|---|---|---|---|---|---|---|---|---|---|---|---|---|
| Fa | 0.1 | 0.15 | 0.2 | 0.25 | 0.3 | 0.35 | 0.4 | 0.45 | 0.5 | 0.55 | 0.6 | 0.65 | 0.7 | 0.75 | 0.8 | 0.85 | 0.9 |
| CI | 1.2 | 1.2 | 1.17 | 1.16 | 1.14 | 1.13 | 1.12 | 1.11 | 1.11 | 1.1 | 1.09 | 1.09 | 1.08 | 1.08 | 1.07 | 1.07 | 1.07 |
| CZAST | | | | | | | | | | | | | | | | | |
| CI | 0.9 | 0.86 | 0.81 | 0.78 | 0.75 | 0.73 | 0.72 | 0.71 | 0.7 | 0.69 | 0.68 | 0.68 | 0.67 | 0.67 | 0.68 | 0.68 | 0.7 |
| Nano | | | | | | | | | | | | | | | | | |

The nanoCombo 1G nanoparticle showed synergistic effects (much of the CI vs Fa curve was below 0.8) on 7 of 8 cell lines after 72 h of treatment (most pronounced on NCI-H226). After 24 h, NP also presented good results, especially on NCI-H226 and NCI-H520 (CI analysis after 25 h was not possible due to low 17-AAG activity).

The nanoCombo 3G nanoparticle showed a synergistic effect (much of the Cl vs Fa curve was below 0.8) on 6 of 8 cell lines after 72 h of treatment (most pronounced on NCI-H226 and NCI-H520). After 24 h, NP presented good results on a limited number of cell lines (8MGBA and NCI-H226).

The nanoparticle, named nanoCombo 4T, showed a synergistic effect (much of the CI vs Fa curve was below 0.8) on 3 of 8 cell lines after 72 h of treatment (NCI-H226, SKMES-1 and NCI-H520). After 24 h, NP presented good results on a limited number of cell lines (NCI-H226).

1. A composition consisting of 17-AAG (tanespimycin) at a concentration of not less than 0.31 nM and staurosporine at a concentration of not less than 0.031 nM for the treatment of melanoma, non-small cell lung cancer, pancreatic cancer, mammary gland tumours or gliomas.

2. The composition according to claim. 2, characterised in that the concentration ratio of 17-AAG to staurosporine is in the range from 100/1 to 1/1.

3. A stable polymer-lipid nanoparticle for the binding and delivery of drugs, particularly those with anticancer properties, characterised in that it is composed of a core containing polylactic-co-glycolic acid (PLGA) in an amount of 16.1 to 30.3% by weight and polyvinyl alcohol (PVA) in an amount of 60.6 to 75.5% by weight, and a core envelope containing a mixture of lipids: 1,2-dipalmitoyl-sn-glycero-3-phosphocholine (DDPC) in an amount ranging from 3.66% to 12.6% by weight, cholesterol in an amount ranging from 1.85 to 6.29% by weight and the ammonium salt of 1,2-distearoyl-sn-glycero-3-phosphoethanolamine-N-[amino (polyethylene glycol)-2000 (DSPE-PEG (2000)NH2] in an amount of 0.15 to 0.5% by weight contains a pharmaceutical composition comprising 17-AAG (tanespimycin) in a concentration of not less than 0.31 nM and staurosporine at a concentration of not less than 0.031 nM.

4. The nanoparticle according to claim 3, characterised in that the concentration ratio of 17-AAG to staurosporine is in the range from 100/1 to 1/1.

5. The nanoparticle according to claim 3, characterised in that the PLGA is a 50:50 copolymer with an Mw of 24 000-38 000 g/mol.

6. The nanoparticle according to claim 3, characterised in that the PVA has a mass Mw of 30 000-70 000 g/mol.
